(19) United States
(12) Patent Application Publication
(10) Pub. No.: US 2004/0076606 A1
(43) Pub. Date: Apr. 22, 2004

(54) Methods of modulating inflammation by administration of interleukin-19 and inhibitors of IL-19 binding

(76) Inventor: Ming-Shi Chang, Tainan (CN)

Correspondence Address: FISH & RICHARDSON PC, 225 FRANKLIN ST, BOSTON, MA, 02110, US

(21) Appl. No.: 10/247,843

(22) Filed: Sep. 14, 2002

(51) Int. Cl. A61K 38/20; C12Q 1/68; C07H 21/04; C12P 21/04; C12N 5/06; C07K 14/54

(52) U.S. Cl. 424/85.2; 435/6; 435/69.52; 435/320.1; 435/325; 530/351; 536/23.5

(57) ABSTRACT

Methods for modulating inflammation using IL-19 polypeptides and inhibitors of IL-19 binding to an IL-19 receptor are disclosed. The present invention also provides the human IL-19 promoter and use of the promoter to detect polymorphisms in the Il-19 promoter region of an individual. Also disclosed are purified and isolated murine IL-19 polynucleotides and polypeptides.

### FIELD OF THE INVENTION

The present invention relates to methods of increasing circulating interleukin-6 and/or TNF-α by administering IL-19, and to methods for decreasing circulating interleukin-6 and/or TNF-α by administering an inhibitor of IL-19 binding to an IL-19 receptor. Treatment of diseases associated with TNF-α or IL-6 expression are also provided. The present invention also provides an human IL-19 promoter sequence and methods for detecting polymorphisms in an IL-119 promoter sequence, and further provides a purified and isolated murine IL-19 polynucleotide and polypeptide.

### BACKGROUND OF THE INVENTION

Interleukin-19 (IL-19) is a member of the IL-10 cytokine family, which includes IL-20, IL-22, IL-24, and IL-26. IL-10 was originally described as a cytokine synthesis inhibitory factor due to its inhibitory effect on production of inflammatory cytokines such as IL-1, tumor necrosis factor-α (TNF-α) and IL-6 (Gesser et al., Proc. Natl. Acad. Sci. USA 94:14620. 1997.; Ding et al., J. Exp. Med. 191:213. 2000). IL-10 has also been deemed an endogenous feedback factor for the down-regulation and control of immune responses and inflammation. In addition, IL-10 has been demonstrated to act as a stimulatory factor for mast cells, B cells, and thymocytes (Go, et al. J Exp. Med. 172:1625. 1990; Thompson-Snipes, et al. J. Exp. Med. 173:507. 1991; Rousset, et al. Proc. Natl. Acad. Sci. USA 89:1890. 1992) as well as be pleiotropic in its ability to act on many other cell types including monocytes/macrophages, T cells, natural killer cells, neutrophils, endothelial cells, and peripheral blood mononuclear cells (PBMC). (de Waal, M. R.. In Cytokine. A. R. Mire-Sluis, and R. Thorpe, eds. Academic Press, San Diego, Calif., p. 151. 1998; de Waal et al. J Exp. Med. 174:1209.1991).

Several new members of the IL-10 family, including IL-19, IL-20, IL-22, MDA-7 (IL-24), and AK155 (IL-26), have only recently been discovered. The IL-19, IL-20, and MDA-7 (IL-24) genes have been mapped to chromosome locus 1q31-³2, where the gene encoding IL-10 is located. Genes encoding the two other IL-10 related cytokines, AK155 (IL-26) and IL-22, are on chromosome 12q15 (Dumoutier, et al. J. Immunol. 167:3545. 2001). Overexpression of IL-20 in transgenic mice has been shown to cause neonatal death as well as skin abnormalities, including aberrant epidermal differentiation (Blumberg, et al. Cell 104:9. 2001). IL-22 was originally identified as an upregulated gene product induced following IL-9 with murine T lymphocytes. Stimulation of HepG2 human hepatoma cells with IL-22 has been shown to upregulate the production of acute phase reactants like serum amyloid A, α1-antichymotrypsin, and haptoglobin (Dumoutier et al. J Immunol. 164:1814. 2000; Dumoutier, et al. Proc. Natl. Acad. Sci. USA 97:10144. 2000). Expression of MDA-7 is up-regulated in wound healing and during the in vitro differentiation of a melanoma cell line (Rich et al. Curr. Biol. 11:R531. 2001; Jiang et al. Oncogene 11:2477. 1995). AKI 55 is known to be induced by transformation of T lymphocytes with herpesvirus saimiri, but its biologic activities and receptor remain unknown (Dumoutier, et al. J Immunol. 167:3545. 2001; Knappe, et al. J Virol. 74:3881. 2000).

One new member of the IL-10 family, IL-19, has recently been identified and the human cDNA isolated and cloned (U.S. Pat. No. 5,985,614; Gallagher et al., Genes Immunol. 1:422. 2000). Very little is known about this cytokine functionally except that IL-19 has been shown to expressed by lipopolysaccharaide-(LPS) or granulocyte/monocyte-colony stimulating factor-(GM-CSF) activated monocytes (Gallagher et al, supra). It has also been reported that IL-19 binds to the IL-20α/β receptor heterodimer and activates STAT-3 phosphorylation and signaling pathway, but the biological effect of activity is still unclear (Dumoutier et al., J Immunol. 2001, supra).

Due to the shared homology between IL-19 and IL-10, it was proposed that IL-19 possesses IL-10-like anti-inflammatory activity, indicating that IL-19 functions in downregulating inflammatory immune responses by inhibiting the production of cytokines such as IFN-γ and TNF-α. Additionally, like IL-10, IL-19 was proposed to act stimulate survival and differentiation of antibody producing B cells. Thus, IL-19 administration was predicted to function as an immunosuppressive therapy to treat diseases mediated by ongoing inflammation including autoimmune diseases, Graft vs Host disease, sepsis, and the like.

Thus there exists a need in the art to identify the biological function of IL-19 and determine its role in modulation of inflammation. Identification of orthologs of IL-19 are also needed to assess the biological role of IL-19 using animal models of human diseases and to develop therapeutics based on these animal models.

### SUMMARY OF THE INVENTION

The present invention relates to methods of modulating inflammation by the administration of soluble IL-19 polypeptide and inhibitors of IL-19 binding to an IL-19 receptor to respectively increase or decrease the levels of inflammatory cytokines in an individual. In a related aspect the invention relates to a purified and isolated polynucleotide encoding a promoter for a human IL-19 gene. In another aspect the invention provides a purified and isolated polynucleotide and polypeptide encoding a murine homolog of human IL-19 and host cells and vectors thereof.

In one embodiment, the invention provides methods for increasing production of IL-6 comprising the step of administering to an individual in need thereof an amount of IL-19 polypeptide effective to increase production of IL-6. In another embodiment, methods for increasing production of TNF-α are provided comprising the step of administering to an individual in need thereof an amount of IL-19 polypeptide effective to increase production of TNF-α. In another embodiment, the invention provides methods for increasing production of reactive oxygen species comprising the step of administering to an individual in need thereof an amount of IL-19 polypeptide effective to increase reactive oxygen species. In a further embodiment, methods are provided for increasing apoptosis comprising the step of administering to an individual in need thereof an amount of IL-19 polypeptide effective to increase apoptosis.

In a related aspect, the invention provides a method of transmembrane signaling comprising the step of stimulating the IL-20α/β receptor. In one embodiment, the method contemplates increasing production of IL-6 in an individual in need thereof comprising the step of stimulating the IL-20α/β receptor effective to increase production of IL-6. In another embodiment, the invention provides a method for increasing production of TNF-α in an individual in need thereof comprising the step of stimulating the IL-20α/β receptor effective to increase production of TNF-α. Further contemplated is a method for increasing production of reactive oxygen species in an individual in need thereof comprising the step of stimulating the IL-20α/β, receptor effective to increase production of reactive oxygen species. An additional embodiment provides a method for increasing apoptosis in an individual in need thereof comprising the step of stimulating the IL-20α/β receptor effective to increase apoptosis. In an additional embodiment, the invention provides methods of transmembrane signaling comprising the step of stimulating the IL-20α/β: receptor and methods of increasing production of IL-6, TNF-α, reactive oxygen species or apoptosis in an individual in need thereof comprising the step of stimulating the IL-20α/β receptor effective to increase production of IL-6, TNF-α, reactive oxygen species or apoptosis, wherein stimulating is by contact with an IL-19 polypeptide.

The invention further provides variants of the IL-19 polypeptide. Preferably, the IL-19 polypeptide variants competitively bind to an IL-19 receptor, preventing the binding of IL-19 and activation of the receptor molecule. The variants of this type include amino acid deletion-, addition-, or substitution-analogs and peptide mimetics, which are easily prepared using techniques well-known in the art.

Also comprehended by the present invention are polypeptides and other non-peptide molecules which specifically bind to IL-19. Preferred binding molecules include antibodies (e.g., monoclonal and polyclonal antibodies, recombinant, chimeric, humanized such as CDR-grafted, human, single chain, catalytic, multi-specific and/or bi-specific, as well as fragments, variants, and/or derivatives thereof), counterreceptors (e.g., membrane-associated and soluble forms) and other ligands (e.g., naturally occurring or synthetic molecules), including those which competitively bind IL-19 in the presence of IL-19 monoclonal antibodies and/or specific counterreceptors. Binding molecules are useful for purification of IL-19 polypeptides and identifying cell types which express IL-19. Binding molecules are also useful for modulating (i.e., inhibiting, blocking or stimulating) in vivo binding and/or signal transduction activities of IL-19.

Biological assays to identify IL-19 binding molecules are also provided, including immobilized ligand binding assays, solution binding assays, scintillation proximity assays, di-hybrid screening assays, and the like.

In vitro assays for identifying antibodies or other compounds that bind to or modulate the activity of IL-19 may involve, for example, immobilizing IL-19 or a natural ligand or binding molecule to which IL-19 binds, detectably labeling the nonimmobilized binding partner, incubating the binding partners together and determining the effect of a test compound on the amount of label bound wherein a reduction in the label bound in the presence of the test compound compared to the amount of label bound in the absence of the test compound indicates that the test agent is an inhibitor of IL-19 binding.

Another type of assay for identifying compounds that modulate the interaction between IL-19 and a ligand involves immobilizing IL-19 or a fragment thereof on a solid support coated (or impregnated with) a fluorescent agent, labeling the ligand with a compound capable of exciting the fluorescent agent, contacting the immobilized IL-19 with the labeled binding molecule in the presence and absence of a putative modulator compound, detecting light emission by the fluorescent agent, and identifying modulating compounds as those compounds that affect the emission of light by the fluorescent agent in comparison to the emission of light by the fluorescent agent in the absence of a modulating compound. Alternatively, the IL-19 ligand may be immobilized and IL-19 may be labeled in the assay.

Yet another method contemplated by the invention for identifying compounds that modulate the interaction between IL-19 and an IL-19 binding molecule involves transforming or transfecting appropriate host cells with a DNA construct comprising a reporter gene under the control of a promoter regulated by a transcription factor having a DNA-binding domain and an activating domain, expressing in the host cells a first hybrid DNA sequence encoding a first fusion of part or all of IL-19 and either the DNA binding domain or the activating domain of the transcription factor, expressing in the host cells a second hybrid DNA sequence encoding part or all of the ligand and the DNA binding domain or activating domain of the transcription factor which is not incorporated in the first fusion, evaluating the effect of a putative modulating compound on the interaction between IL-19 and the ligand by detecting binding of the ligand to IL-19 in a particular host cell by measuring the production of reporter gene product in the host cell in the presence or absence of the putative modulator, and identifying modulating compounds as those compounds altering production of the reported gene product in comparison to production of the reporter gene product in the absence of the modulating compound. Presently preferred for use in the assay are the lexA promoter, the lexA DNA binding domain, the GAL4 transactivation domain, the lacZ reporter gene, and a yeast host cell.

Further contemplated by the invention are methods for ameliorating a condition associated with decreased levels of IL-6, TNF-α, reactive oxygen species, or apoptosis comprising the step of administering to an individual an amount of IL-19 polypeptide effective to increase levels of IL-6, TNF-α, reactive oxygen species, or apoptosis. In one embodiment, the methods further comprise administering other therapeutic compounds in conjunction with IL-19 polypeptides. The invention further provides for IL-19 polypeptides in a pharmaceutically acceptable carrier solution conventionally used to deliver therapeutics or imaging agents.

In a related aspect, the invention provides a method for modulating inflammation comprising the step of administering to an individual in need thereof an amount of an inhibitor of IL-19 binding to an IL-19 receptor effective to modulate inflammation. In one embodiment, the method for modulating inflammation comprising the step of administering an inhibitor of IL-19 binding to an IL-19 receptor is a method wherein the production of IL-6 is decreased by administering the inhibitor. In another embodiment, the method for modulating inflammation comprising the step of administering an inhibitor of IL-19 binding to an IL-19 receptor is a method wherein the production of TNF-α is decreased by administering the inhibitor. In an additional embodiment, methods are provided for decreasing production of reactive oxygen species comprising the step of administering to an individual in need thereof an amount of an inhibitor of IL-19 binding to an IL-19 receptor effective to decrease reactive oxygen species. In a further embodiment, the invention provides methods for decreasing apoptosis comprising the step of administering to an individual in need thereof an amount of an inhibitor of IL-19 binding to an IL-19 receptor effective to decrease apoptosis.

Also contemplated by the invention are methods for ameliorating a condition associated with increased levels of IL-6, TNF-α, reactive oxygen species, or apoptosis comprising the step of administering to an individual an effective amount of an inhibitor of IL-19 binding to an IL-19 receptor effective to decrease levels of IL-6, TNF-α, reactive oxygen species, or apoptosis. In one embodiment, the inhibitor of IL-19 binding to an IL-19 receptor is selected from the group consisting of an IL-19 blocking antibody or an antigen binding fragment of an IL-19 blocking antibody, a soluble form of an IL-19 receptor, soluble receptor peptides, an IL-19 receptor blocking antibody or antigen binding fragments of an IL-19 receptor blocking antibody and polypeptides and other non-peptide molecules which specifically bind to IL-19. Also contemplated are compositions wherein the inhibitor of IL-19 binding to an IL-19 receptor is in a pharmaceutically acceptable carrier.

In another embodiment, the invention provides a method for modulating inflammation wherein the IL-19 polypeptide and/or the inhibitor of IL-19 binding to an IL-19 receptor is administered in combination with other therapeutic compounds for the treatment prevention or amelioration of a disease, condition, or disorder requiring the modulation of inflammation.

In a related aspect, the inventions provides a purified and isolated polynucleotide encoding a promoter for a human IL-19. In one embodiment the human IL-19 promoter is set out in SEQ. ID NO.: 1. Use of such promoter sequences are particularly desirable in instances, for example gene transfer, which can specifically require heterologous gene expression in a limited environment. The invention also comprehends vectors comprising promoters of the invention, as well as chimeric gene constructs wherein the promoter of the invention is operatively linked to a heterologous polynucleotide sequence and a transcription termination signal.

Also provided is a method for identifying polymorphisms in an IL-19 promoter region of an individual, comprising comparing the IL-19 promoter region in the individual to the IL-19 promoter of SEQ. ID NO.: 1, wherein a difference in the nucleotide sequence of the IL-19 promoter is indicative of a polymorphism in the IL-19 promoter region of the individual. The invention further provides a method of identifying polymorphisms wherein the comparison is carried out by restriction enzyme mapping, PCR analysis, DNA hybridization. In one embodiment, the comparison is carried out using DNA hybridization. In another embodiment, the DNA hybridization is performed wherein an IL-19 promoter from an individual is hybridized to a set of fragments taken from SEQ. ID NO.: 1, said fragments consisting of at least 10 nucleotides, at least 15 nucleotides and at least 20 nucleotides. Further contemplated by the invention is a method wherein the set of fragments taken from SEQ. ID NO.: 1 overlap by at least one nucleotide.

Another aspect of the invention provides for a purified and isolated polynucleotide (e.g., DNA and RNA transcripts, both sense and anti sense strands) encoding murine IL-19 and variants thereof (i.e., deletion, addition or substitution analogs). The invention further provides a purified and isolated murine IL-19 polypeptide having the sequence set out in SEQ. ID NO.: 6. and a polynucleotide encoding the IL-19 polypeptide of SEQ. ID NO.: 6. The invention further contemplates an anti-sense polynucleotide which specifically hybridizes to the polynucleotide encoding the polypeptide of SEQ. ID NO.: 6. In a related embodiment, the invention provides a murine IL-19 polynucleotide having an IL-19 protein coding region set forth in SEQ ID NO: 6 and a polypeptide encoded by said polynucleotide.

In an additional embodiment, the invention provides a purified and isolated murine polynucleotide encoding a murine IL-19 amino acid sequence selected from the group consisting of: a polynucleotide encoding a purified and isolated murine IL-19 polypeptide having the sequence set out in SEQ. ID NO.: 6 wherein the polynucleotide has an IL-19 protein coding sequence set out in SEQ. ID NO.: 5; a polynucleotide which hybridizes under stringent conditions to the protein coding portion of the polynucleotide having an IL-19 protein coding sequence set out in SEQ. ID NO.: 6; and a polynucleotide which is at least 85%, at least 90%, at least 95%, at least 96%, at least 97%, at least 98%, at least 99% homologous to the polypeptide coding sequence set out in SEQ. ID NO.: 5.

The invention further contemplates a polypeptide of the invention encoded by a purified and isolated murine polynucleotide encoding a murine IL-19 amino acid sequence selected from the group consisting of: a polynucleotide encoding a purified and isolated murine IL-19 polypeptide having the sequence set out in SEQ. ID NO.: 6 wherein the polynucleotide has an IL-19 protein coding sequence set out in SEQ. ID NO.: 5; a polynucleotide which hybridizes under stringent conditions to the protein coding portion of the polynucleotide having an IL-19 protein coding sequence set out in SEQ. ID NO.: 6 and a polynucleotide which is at least 85%, at least 90%, at least 95%, at least 96%, at least 97%, at least 98%, at least 99% homologous to the polypeptide coding sequence set out in SEQ. ID NO.: 5

Also provided are recombinant plasmid and viral DNA expression constructs comprising a polynucleotide encoding a murine IL-19 amino acid sequence selected from the group consisting of: a polynucleotide encoding a purified and isolated murine IL-19 polypeptide having the sequence set out in SEQ. ID NO.: 6 wherein the polynucleotide has an IL-19 protein coding sequence set out in SEQ. ID NO.: 5; a polynucleotide which hybridizes under stringent conditions to the protein coding portion of the polynucleotide having an IL-19 protein coding sequence set out in SEQ. ID NO.: 6 and a polynucleotide which is at least 85%, at least 90%, at least 95%, at least 96%, at least 97%, at least 98%, at least 99% homologous to the polypeptide coding sequence set out in SEQ. ID NO.: 5. Further provided are host cells comprising the polynucleotide of the invention. Prokaryotic or eukaryotic host cells transformed or transfected with polynucleotides of the invention are contemplated. The invention further provides a method of producing an IL-19 polypeptide comprising growing the host cell of above under conditions that permit expression of an IL-19 polypeptide.

Host cells of the invention include any cell type capable of expressing IL-19 and IL-19 binding proteins. In a preferred embodiment, the host cells are of either mammal, insect or yeast origin. In another aspect, the host cell is a yeast cell, selected from various strains, including S. cerevisiae, S.pombe, K.lactis, P.pastoris, S.carlsbergensis and C.albicans. Mammalian host cells of the invention include Chinese hamster ovary (CHO), COS, HeLa, 3T3, CV1, LTK, 293T3, Rat1, PC12 or any other cell line of human or rodent origin routinely used in the art. Insect host cell lines include SF9 cells. Additional plasmids and host cells available for use are described below.

Also provided are purified and isolated murine IL-19 polypeptides, fragments and variants thereof. A preferred IL-19 polypeptide is as set forth in SEQ ID NO: 6. IL-19 products of the invention may be obtained as isolates from natural sources, but, along with IL-19 variant products, are also produced by recombinant procedures using host cells of the invention. Completely glycosylated, partially glycosylated and wholly de-glycosylated forms of the IL-19 polypeptide may be generated by varying the host cell selected for recombinant production and/or post-isolation processing. Variant IL-19 polypeptides of the invention may comprise water soluble and insoluble IL-19 polypeptides and analogs wherein one or more of the amino acids are deleted or replaced: (1) without loss, and preferably with enhancement, of one or more biological activities or immunological characteristics specific for IL-19; or (2) with specific disablement of a particular ligand/receptor binding or signaling function. In one embodiment, the variant or analog IL-19 polypeptides possesses at least 85%, at least 90%, at least 95%, at least 96%, at least 97%, at least 98% or at least 99% percent identity to the amino acid sequence set out in SEQ. ID NO.: 2.

The invention also contemplates an antibody specifically immunoreactive with the IL-19 polypeptide of the invention which is encoded by the polynucleotide encoding a murine IL-19 amino acid sequence which hybridizes under stringent conditions to the protein coding portion of SEQ. ID NO.: 5 or polypeptide encoded by a polynucleotide which is a least 85%, at least 90%, at least 95%, at least 96%, at least 97%, at least 98%, at least 99% homologous to the polypeptide coding sequence set out in SEQ. ID NO.: 5. In one embodiment, the antibody is a monoclonal antibody.

The invention further provides a method for detecting a polypeptide of the invention in a sample, comprising contacting the sample with a compound that binds to and forms a complex with the polypeptide under conditions sufficient to form the complex; and detecting the complex, so that if a complex is detected, the polypeptide of the invention is detected. The test samples of the present invention include cells, protein or membrane extracts of cells, or biological fluids such as sputum, blood, serum, plasma, or urine.

In a related aspect the invention provides a method for identifying a compound that binds to a polypeptide of the invention, comprising contacting a compound with the polypeptide of the invention under conditions sufficient to form a polypeptide/compound complex; and identifying the compound in the complex.

### BRIEF DESCRIPTION OF THE FIGURES

FIG. 1 is a comparison of mouse and human IL-19 amino acid sequences. Identical amino acid sequences are indicated by |. Similar amino acid sequences are indicated by :. The six conserved cysteines are in bold type. Potential N-linked glycosylation sites are indicated by ***. Signal peptide cleavage sites is indicated by ↓. The location of mouse IL-19 introns are shown by ▾.

### DETAILED DESCRIPTION OF THE INVENTION

The present invention relates to uses for the cytokine IL-19 for the induction of inflammatory cytokines and the use of an inhibitor of IL-19 binding to an IL-19 receptor in the downregulation of inflammation and reactive oxygen species.

Definitions

As utilized in accordance with the present disclosure, the following terms unless otherwise indicated, shall be understood to have the following meanings:

The terms “effective amount” and “therapeutically effective amount” refer to the amount of a IL-19 polypeptide or IL-19 nucleic acid molecule used to support an observable level of one or more biological activities of the IL-19 polypeptides as set forth herein.

The term “expression vector” refers to a vector which is suitable for use in a host cell and contains nucleic acid sequences which direct and/or control the expression of inserted heterologous nucleic acid sequences. Expression includes, but is not limited to, processes such as transcription, translation, and RNA splicing, if introns are present.

The term “host cell” is used to refer to a cell which has been transformed, or is capable of being transformed with a nucleic acid sequence and then of expressing a selected gene of interest. The term includes the progeny of the parent cell, whether or not the progeny is identical in morphology or in genetic make-up to the original parent, so long as the selected gene is present.

The term “identity” as known in the art, refers to a relationship between the sequences of two or more polypeptide molecules or two or more nucleic acid molecules, as determined by comparing the sequences. In the art, “identity” also means the degree of sequence relatedness between nucleic acid molecules or polypeptides, as the case may be, as determined by the match between strings of two or more nucleotide or two or more amino acid sequences. “Identity” measures the percent of identical matches between the smaller of two or more sequences with gap alignments (if any) addressed by a particular mathematical model or computer program (i.e., “algorithms”).

The term “isolated nucleic acid molecule” refers to a nucleic acid molecule of the invention that (1) has been separated from at a least about 50 percent of proteins, lipids , carbohydrates or other materials with which it is naturally found when total DNA is isolated for the source cells, (2) is not linked to all or a portion of a polynucleotide to which the “isolated nucleic acid molecule” is linked in nature, (3) is operably linked to a polynucleotide which it is not linked to in nature, or (4) does not occur in nature as part of a larger polynucleotide sequence. Preferably, the isolated nucleic acid molecule of the present invention is substantially free from at least one contaminating nucleic acid molecule with which it is naturally associated. Preferably, the isolated nucleic acid molecule of the present invention is substantially free from any other contaminating nucleic acid molecule(s) or other contaminants that are found in its natural environment which would interfere with its use in polypeptide production or its therapeutic, diagnostic, prophylactic or research use.

The term “isolated polypeptide” refers to a polypeptide of the present invention that (1) has been separated from at least about 50 percent of polynucleotides, lipids, carbohydrates or other materials with which it is naturally found when isolated from the cell source, (2) is not linked (by covalent or noncovalent interaction) to all or a portion of a polypeptide to which the “isolated polypeptide” is linked to in nature, (3) is operably linked (by covalent or noncovalent interaction) to a polypeptide with which it is not linked in nature, or (4) does not occur in nature. Preferably is free from at least one contaminating polypeptide or other contaminants that are found in its natural environment. Preferably, the isolated polypeptide is substantially free from any other contaminating polypeptides or other contaminants that are found in its natural environment which would interfere with its therapeutic, diagnostic, prophylactic or research use.

The term “stringent” is used to refer to conditions that are commonly understood in the art as stringent. Stringent conditions can include highly stringent conditions (i.e., hybridization to filter-bound DNA in 0.5 M NaHPO4, 7% sodium dodecyl sulfate (SDS), 1 mM EDTA at 65° C., and washing in 0.1×SSC/0.1% SDS at 68° C.), and moderately stringent conditions (i.e., washing in 0.2×SSC/0.1% SDS at 42° C.).

In instances of hybridization of deoxyoligonucleotides, additional exemplary stringent hybridization conditions include washing in 6× SSC/0.05% sodium pyrophosphate at 37° C. (for 14-base oligonucleotides), 48° C. (for 17-base oligonucleotides), 55°C. (for 20-base oligonucleotides), and 60° C. (for 23-base oligonucleotides).

The term “pharmaceutically acceptable carrier” or “physiologically acceptable carrier” as used herein refers to one or more formulation materials suitable for accomplishing or enhancing the delivery of the IL-19 polypeptide, IL-19 nucleic acid molecule or inhibitor of IL-19 binding to an IL-19 receptor as a pharmaceutical composition.

The terms “IL-19 polypeptide” and “IL-19 composition” are used interchangeably herein. The terms refer to any soluble IL-19 polypeptide or fragment thereof that retains natural IL-19 function and binding to the IL-19 receptor. A “variant” of a molecule such as IL-19 polypeptide is meant to refer to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

A variant of the IL-19 polypeptide also includes polypeptide variants which competitively bind to an IL-19 receptor, preventing the binding of IL-19 and activation of the receptor molecule. The variants of this type include amino acid deletion-, addition-, or substitution-analogs and peptide mimetics.

Apart from the foregoing considerations, it will be understood that innumerable conservative amino acid substitutions can be performed to a wildtype IL-19 sequence which result in a polypeptide that retains IL-19 biological activities, especially if the number of such substitutions is small. By “conservative amino acid substitution” is meant substitution of an amino acid with an amino acid having a side chain of a similar chemical character. Similar amino acids for making conservative substitutions include those having an acidic side chain (glutamic acid, aspartic acid); a basic side chain (arginine, lysine, histidine); a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine, threonine). Addition or deletion of one or a few internal amino acids without destroying IL-19 biological activities also is contemplated.

Derivatives, analogues, or peptides may have enhanced or diminished biological activities in comparison to native IL-19, depending on the particular application. IL-19 related derivatives, analogues, peptides and peptide mimetics of the invention may be produced by a variety of means well-known in the art. Procedures and manipulations at the genetic and protein levels are within the scope of the invention. Peptide synthesis, which is standard in the art, may be used to obtain IL-19 peptides. At the protein level, numerous chemical modifications may be used to produce IL-19-like derivatives, analogues, or peptides by techniques known in the art, including but not limited to specific chemical cleavage by endopeptidases (e.g. cyanogen bromides, trypsin, chymotrypsin, V8 protease, and the like) or exopeptidases, acetylation, formylation, oxidation, etc.

The term “inhibitor of IL-19 binding to an IL-19 receptor” refers to a molecule or molecules having specificity for an IL-19 polypeptide wherein the binding of the inhibitor inhibits IL-19 biological function. Inhibitors include IL-19 blocking antibodies, such as polyclonal antibodies, monoclonal antibodies (mAbs), chimeric antibodies, CDR-grafted antibodies, anit-idiotypic (anti-Id) antibodies to antibodies that can be labeled in soluble or bound forms, as well as antigen-binding fragments, regions, or derivatives thereof which are provided by known techniques, including, but not limited to enzymatic cleavage, peptide synthesis, or recombinant techniques. Inhibitors also include soluble forms of an IL-19 receptor, soluble IL-19 antigen binding fragments of the receptors, as well as other small molecules (polypeptides, polynucleotides, or chemical agents) which interfere with IL-19 binding to its receptor.

As used herein, the terms, “specific” and “specificity” refer to the ability of the antagonist to bind to IL-19 polypeptides and not to bind to non-IL-19 polypeptides. It will be appreciated, however, that the antagonists may also bind orthologs of the polypeptide as set forth in SEQ ID NO: 6, that is, interspecies versions thereof, such as human and rat polypeptides.

IL-19 polypeptides, fragments, variants, and derivatives may be used to prepare IL-19 polypeptide compositions or inhibitors of IL-19 binding to an IL-19 receptor using methods known in the art. Thus, antibodies and antibody fragments that bind IL-19 polypeptides are within the scope of the present invention. Antibody fragments include those portions of the antibody which bind to an epitope on the IL-19 polypeptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions. These antibodies may be, for example, polyclonal monospecific polyclonal, monoclonal, recombinant, chimeric, humanized, human, single chain, and/or bispecific.

Differences in the nucleic acid sequence may result in conservative and/or non-conservative modifications of the amino acid sequence relative to the amino acid sequence of SEQ ID NO: 6 and human IL-19.

Conservative modifications to the amino acid sequence of SEQ ID NO: 6 (and the corresponding modifications to the encoding nucleotides) will produce IL-19 polypeptides having functional and chemical characteristics similar to those of naturally occurring IL-19 polypeptide. In contrast, substantial modifications in the functional and/or chemical characteristics of IL-19 polypeptides may be accomplished by selecting substitutions in the amino acid sequence of SEQ ID NO: 6 that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain. Addition or deletion of one or a few internal amino acids without destroying IL-19 biological activities also is contemplated.

Desired amino acid substitutions (whether conservative or non-conservative) can be determined by those skilled in the art at the time such substitutions are desired. For example, amino acid substitutions can be used to identify important residues of the IL-19 polypeptide, or to increase or decrease the affinity of the IL-19 polypeptides described herein.

Preferred derivatives, analogs, and peptides are those which retain IL-19 biological activity.

IL-19 and Inflammation

Based on sequence similarities, it has been predicted that IL-19 biological activity is similar to that of IL-10, and is involved in immunosuppression and downregulation of the immune response. For example, U.S. patent application Ser. No. 2002/0032311 and related PCT application W098/08870 disclose methods for treating an individual in need of a decreased level of IFN-γ, TNF-α and IL-6 activity by administering an IL-19 composition.

The present invention, however, arises from the demonstration that IL-19 does not function with the predicted IL-10-like, immunosupressant activity, but rather is an activator of inflammatory cytokines IL-6 and TNF-α, increases the production of reactive oxygen species and induces apoptosis in cells expressing the receptor. The effects of inducing secretion of inflammatory cytokines can play a significant role in modulating downstream signaling effects in many different biological areas.

Moreover, analysis of single nucleotide polymorphisms detected within the IL-10 promoter region indicated that an amino acid change at residue-1082, residue-819, or residue-592 has been associated with the development of autoimmune diseases such as rheumatoid arthritis and systemic lupus erythematosus (Hajeer, et al. Scand. J. Rheumatol. 27:142-5. 1998; Gibson, et al. J Immunol. 166:3915-22.2001). Thus, the IL-19 promoter region is a useful mechanism by which IL-19 cytokine production, as well as the effects downstream of IL-19 produciton, can be regulated, as well as a method by which aberrant regulation is detectable.

Interleukin-6 (IL-6) is the end-product of a cytokine signaling cascade and is secreted by specialized immune cells during inflammation. It has an influence on many biological functions, including differentiation, stimulation, and activation of immune cells, or other cells of neuroendocrine origin. Changes in the levels of expression of this cytokine and its receptor have been observed during chronic inflammatory disease, and have been associated with tumorigenesis. Recent studies also suggest that IL-6 is involved in the development of lung cancer.

TNF-α, a potent inflammatory cytokine which asserts its function on macrophage cells, has been implicated as a key mediator in many inflammatory pathologies, including autoimmune diseases (arthritis, multiple sclerosis, and type I diabetes), as well as the acting as the key factor in septic shock. TNF-α secretion by inflammatory T cells and activated macrophages induces macrophages to secrete other inflammatory signals as well as damaging oxygen reactive species. The downstream effects of TNF-α result in activation of the vascular endothelium and increased vascular permeability, leading to greater immune cell infiltration to the site of inflammation, thus perpetuating the cycle of inflammation. TNF-α along with IL-1 and IL-6 produce fever and increased body temperature in response to bacterial infection and also activate the liver to produce acute phase proteins in response to bacterial infection.

Reactive oxygen species (ROS), one of the defense mechanisms produced by activated macrophages, have also been implicated as factors in several widespread diseases including Alzheimer's disease, Parkinson's disease, myocardial infarction, atherloslcerosis, autoimmune diseases, sunburn, aging, and radiation injury. Reactive oxygen species are those which contain free oxygen radicals formed during the metabolism of oxygen, and include, for example, O₂(—), OH and H₂O₂. Oxidative stress results from an imbalance of radical production and radical scavenging (mediated chiefly by superoxide dismutase (SOD) and glutathione peroxidase). Free oxygen radicals exert their deleterious effects predominantly on lipid fatty acid side chains, removing electrons from these fatty acids to produce stable oxygen species, but producing another free radical in the process. Eventually, a fatty acid free radical will covalently join with another fatty acid radical which together exert a damaging effect on cell membrane integrity.

ROS are necessary, however, for the immune system defense against bacterial infection. For instance, chronic granulomatous disease (CGD), a disorder where individuals cannot adequately defend against bacterial infections, results from a mutation in a subunit of NADPH oxidase, which is important for oxygen radical production by activated macrophages (Goldblatt D. Expert Opin Pharmacother. 2002. 3:857-63). Because the host defenses are so weakened by lacking a primary form of natural defenses, CGD patients are routinely secondarily infected by bacterial or fungal pathogens.

Apoptosis, or programmed cell death, functions in maintaining normal tissue homeostasis in a variety of physiological processes including embryonic development, immune cell regulation, normal cellular turnover and the programmed cell death of cancer cells. Thus, the dysregulation or loss of regulated apoptosis can lead to a variety of pathological disease states. For example, the loss of apoptosis can lead to the pathological accumulation of self-reactive lymphocytes as observed in many autoimmune diseases. Inappropriate regulation of apoptosis also can lead to the accumulation of virally infected cells and of hyperproliferative cells, such as neoplastic or tumor cells. Inappropriate activation of apoptosis can contribute to a variety of diseases such as AIDS, neurodegenerative diseases and ischemic injury.

Dysregulation of apoptosis has been implicated in numerous diseases such as cardiovascular diseases, especially those which are associated with apoptosis of endothelial cells, degenerative liver disease, multiple sclerosis, rheumatoid arthritis, hematological disorders including lymphoma, leukemia, aplastic anemia, and myelodysplastic syndrome, osteoporosis, polycystic kidney disease, AIDS, myelodysplastic syndromes, aplastic anemia and baldness.

Neurodegenerative disorders affected include Alzheimer's disease, Parkinson's disease, Huntington's disease, amyotrophic lateral sclerosis (ALS), cerebellar degeneration, stroke, traumatic brain injury, central nervous system (CNS) ischemic reperfusion injury including neonatal hypoxic-ischemic brain injury or myocardial ischemic-reperfusion injury, injury caused by hypoxia.

Inflammatory disease states include systemic inflammatory conditions and conditions associated locally with migration and attraction of monocytes, leukocytes and/or neutrophils. Inhibition of chemotaxis or chemokine activity may be useful to ameliorate pathologic inflammatory disease states. Inflammation may result from infection with pathogenic organisms (including gram-positive bacteria, gram-negative bacteria, viruses, fungi, and parasites such as protozoa and helminths), transplant rejection (including rejection of solid organs such as kidney, liver, heart, lung or cornea, as well as rejection of bone marrow transplants including graft-versus-host disease (GVHD)), or from localized chronic or acute autoimmune or allergic reactions. Autoimmune diseases include acute glomerulonephritis; rheumatoid or reactive arthritis; chronic glomerulonephritis; inflammatory bowel diseases such as Crohn's disease, ulcerative colitis and necrotizing enterocolitis; granulocyte transfusion associated syndromes; inflammatory dermatoses such as contact dermatitis, atopic dermatitis, psoriasis; systemic lupus erythematosus (SLE), autoimmune thyroiditis, multiple sclerosis, some forms of diabetes, or any other autoimmune state where attack by the subject's own immune system results in pathologic tissue destruction. Allergic reactions include allergic asthma, chronic bronchitis, allergic rhinitis, and acute and delayed hypersensitivity. Systemic inflammatory disease states include inflammation associated with trauma, bums, reperfusion following ischemic events (e.g., thrombotic events in heart, brain, intestines or peripheral vasculature, including myocardial infarction and stroke), sepsis, ARDS or multiple organ dysfunction syndrome. Inflammatory cell recruitment also occurs in atherosclerotic plaques.

Viral infections that may be treated include infections caused by herpesviruses (including CMV, HSV-1, HSV-2, VZV, EBV, HHV-6, HHV-7 and HHV-8), paramyxoviruses (including parainfluenza, mumps, measles, and respiratory syncytial virus (RSV)), picomaviruses (including enteroviruses and rhinoviruses), togaviruses, coronaviruses, arenaviruses, bunyaviruses, rhabdoviruses, orthomyxoviruses (including influenza A, B and C viruses), reoviruses (including reoviruses, rotaviruses and orbiviruses), parvoviruses, adenoviruses, hepatitis viruses (including A, B, C, D and E) and retroviruses (including HTLV and HIV). Treatment of both acute and chronic infections is contemplated.

Examples of pathological conditions resulting from increased cell survival due to dysregulation of apoptosis include cancers such as lymphomas, carcinomas and hormone-dependent tumors (e.g., breast, prostate or ovarian cancer). Abnormal cellular proliferation conditions or cancers that may be treated in either adults or children include solid-phase tumors/malignancies, locally advanced tumors, human soft tissue sarcomas, metastatic cancer, including lymphatic metastases, blood cell malignancies including multiple myeloma, acute and chronic leukemias and lymphomas, head and neck cancers including mouth cancer, larynx cancer and thyroid cancer, lung cancers including small-cell carcinoma and non-small-cell cancers, breast cancers including small-cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastoma, astrocytic brain tumors, gliomas, metastatic tumor cell invasion in the central nervous system, bone cancers including osteomas, skin cancers including malignant melanoma, tumor progression of human skin keratinocytes, squamous cell carcinoma, basal cell carcinoma, hemangiopericytoma and Karposi's sarcoma.

Modulation of any of the above-conditions by the administration of IL-19 compositions or an inhibitor of IL-19 binding to an IL-19 receptor is contemplated by the invention.

Formulation Of Pharmaceutical Compounds

The IL-19 and inhibitor of IL-19 binding to an IL-19 receptor are administered in pharmaceutically acceptable carriers as described below. Pharmaceutical compounds include pharmaceutically acceptable salts, particularly where a basic or acidic group is present in a compound. For example, when an acidic substituent, such as —COOH, is present, the ammonium, sodium, potassium, calcium and the like salts, are contemplated as preferred embodiments for administration to a biological host. When a basic group (such as amino or a basic heteroaryl radical, such as pyridyl) is present, then an acidic salt, such as hydrochloride, hydrobromide, acetate, maleate, pamoate, phosphate, methanesulfonate, p-toluenesulfonate, and the like, is contemplated as a preferred form for administration to a biological host.

Similarly, where an acid group is present, then pharmaceutically acceptable esters of the compound (e.g., methyl, tert-butyl, pivaloyloxymethyl, succinyl, and the like) are contemplated as preferred forms of the compounds, such esters being known in the art for modifying solubility and/or hydrolysis characteristics for use as sustained release or prodrug formulations.

In addition, some compounds may form solvates with water or common organic solvents. Such solvates are contemplated as well.

Pharmaceutical IL-19 and inhibitors of IL-19 binding to an IL-19 receptor can be used directly to practice materials and methods of the invention, but in preferred embodiments, the compounds are formulated with pharmaceutically acceptable diluents, adjuvants, excipients, or carriers. The phrase “pharmaceutically or pharmacologically acceptable” refer to molecular entities and compositions that do not produce adverse, allergic, or other untoward reactions when administered to an animal or a human, e.g., orally, topically, transdermally, parenterally, by inhalation spray, vaginally, rectally, or by intracranial injection. (The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well.) Generally, this will also entail preparing compositions that are essentially free of pyrogens, as well as other impurities that could be harmful to humans or animals. The term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art.

The pharmaceutical compositions containing the IL-19 polypeptides and inhibitors of IL-19 binding to an IL-19 receptor described above may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelating capsules wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compositions may also be in the form of suppositories for rectal administration of the PTPase modulating compound. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols, for example.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Administration and Dosing

Methods of the invention include a step of polypeptide administration to a human or animal. Polypeptides are administered in any suitable manner using an appropriate pharmaceutically-acceptable vehicle, e.g., a pharmaceutically-acceptable diluent, adjuvant, excipient or carrier. The composition to be administered according to methods of the invention preferably comprises (in addition to the polynucleotide or vector) a pharmaceutically-acceptable carrier solution such as water, saline, phosphate-buffered saline, glucose, or other carriers conventionally used to deliver therapeutics or imaging agents.

The “administering” step that is performed according to the present invention is performed using any medically-accepted means for introducing a therapeutic directly or indirectly into a mammalian subject, including but not limited to injections (e.g., intravenous, intramuscular, subcutaneous, or catheter); oral ingestion; intranasal or topical administration; and the like. In one aspect, the therapeutic composition is delivered to the patient at multiple sites. The multiple administrations are rendered simultaneously or are administered over a period of several hours. In certain cases it is beneficial to provide a continuous flow of the therapeutic composition. Additional therapy may be administered on a period basis, for example, daily, weekly or monthly.

Polypeptides for administration are formulated with uptake or absorption enhancers to increase their efficacy. Such enhancer include for example, salicylate, glycocholate/linoleate, glycholate, aprotinin, bacitracin, SDS caprate and the like. See, e.g., Fix (J. Pharm. Sci., 85(12) 1282-1285, 1996) and Oliyai and Stella (Ann. Rev. Pharmacol. Toxicol., 32:521-544, 1993).

The amount of peptide in a given dosage will vary according to the size of the individual to whom the therapy is being administered, as well as the characteristics of the disorder being treated such as condition, age of patient and severity of disorder. In exemplary treatments, the dosage is administered at about 50 mg/day, 75 mg/day, 100 mg/day, 150 mg/day, 200 mg/day, or 250 mg/day. These concentrations are administered as a single dosage form or as multiple doses. Standard dose-response studies, first in animal models and then in clinical testing, reveal optimal dosages for particular disease states and patient populations. Calculation of doses is routine in the art.

It will also be apparent that dosing should be modified if traditional therapeutics are administered in combination with therapeutics of the invention. For example, treatment of cancer using traditional chemotherapeutic agents or radiation, in combination with methods of the invention, is contemplated.

Therapeutic Uses

A non-exclusive list of uses and treatments for the IL-19 polypeptides and inhibitors of IL-19 binding to an IL-19 receptor of the invention includes: the treatment or prevention of inflammatory disease, autoimmune disease, diseases related to production of reactive oxygen species (ROS), and diseases related to aberrant apoptosis of cells. The inhibitors of IL-19 binding to an IL-19 receptor of the invention are also useful for inhibiting formation of ROS, limiting secretion of inflammatory cytokine and limiting apoptosis.

For example, the invention contemplates treating, preventing, or ameliorating a disease, condition, or disorder associated with increased levels of inflammatory indications comprising the step of administering to a individual an effective amount of an inhibitor of IL-19 binding to an IL-19 receptor, wherein the disease is chosen from the group comprising Alzheimer's disease, myocardial infarction, atherosclerosis, Parkinson's Disease, H. pylori mediated ulcers, autoimmune disease, and septic shock Additionally, the method of the invention includes a method for treating, preventing, or ameliorating a disease, condition, or disorder associated with decreased levels of inflammatory indications comprising the step of administering to an individual an effective amount of IL-19 polypeptide, wherein the disease is chosen from the group comprising chronic granulomatous disease, cancer or AIDS.

As contemplated by the present invention, an IL-19 polypeptide, agonist or an inhibitor of IL-19 binding to an IL-19 receptor thereof may be administered as an adjunct to other therapy and also with other pharmaceutical agents suitable for the indication being treated. An IL-19 polypeptide and any of one or more additional therapies or pharmaceutical agents may be administered separately, sequentially, or simultaneously.

In a specific embodiment, the present invention is also directed to the use of an IL-19 polypeptide or an inhibitor of IL-19 binding to an IL-19 receptor molecule in combination (pretreatment, post-treatment or concurrent treatment) with any of one or more existing therapies for treatment and modulation of inflammation.

Animal Models

Possession of non-human IL-19 DNA sequences permits development of animal models (including, for example, transgenic models) of the human system.

Identification of additional cell types which express IL-19 may have significant ramifications for development of therapeutic and prophylactic agents. It is anticipated that the products of the invention related to IL-19 can be employed in the treatment of diseases wherein monocytes/macrophages are an essential element of the disease process. Animal models for many pathological conditions associated with macrophage activity have been described in the art. For example, in mice, macrophage recruitment to sites of both chronic and acute inflammation is reported by Jutila, et al., J Leukocyte Biol. 54:30-39 (1993). In rats, Adams, et al., [Transplantation 53:1115-1119(1992) and Transplantation 56:794-799 (1993)] describe a model for graft arteriosclerosis following heterotropic abdominal cardiac allograft transplantation. Rosenfeld, et al., [Arteriosclerosis 7:9-23 (1987) and Arteriosclerosis 7:24-34 (1987)] describe induced atherosclerosis in rabbits fed a cholesterol supplemented diet. Hanenberg, et al., [Diabetologia 32:126-134 (1989)] report the spontaneous development of insulin-dependent diabetes in BB rats. Yamada et al., [Gastroenterology 104:759-771 (1993)] describe an induced inflammatory bowel disease, chronic granulomatous colitis, in rats following injections of streptococcal peptidoglycan-polysaccharide polymers. Cromartie, et al., [J. Exp.Med. 146:1585-1602 (1977)] and Schwab, et al., [Infection and Immunity 59:4436-4442 (1991)] report that injection of streptococcal cell wall protein into rats results in an arthritic condition characterized by inflammation of peripheral joints and subsequent joint destruction. Finally, Huitinga, et al., [Eur. J. Immunol 23:709-715 (1993) describe experimental allergic encephalomyclitis, a model for multiple sclerosis, in Lewis rats. In each of these models, IL-19 antibodies, other IL-19 binding proteins, or soluble forms of IL-19 receptor are utilized to attenuate the disease state, presumably through inactivation of macrophage activity.

Nucleic Acid Molecules

Recombinant DNA methods used herein are generally those set forth in Sambrook et al., Molecular Cloning. A Laboratory Manual, Cold Spring Harbor Laboratory Press, Cold Spring Harbor, N.Y. (1989), and/or Ausubel et al., eds., Current Protocols in Molecular Biology, Green Publishers Inc. and Wiley and Sons, NY (1994). The present invention provides for nucleic acid molecules as described herein and methods for obtaining the molecules.

A gene or cDNA encoding a IL-19 polypeptide or fragment thereof may be obtained by hybridization screening of a genomic or cDNA library, or by PCR amplification. Where a gene encoding the amino acid sequence of an IL-19 polypeptide has been identified from one species, all or a portion of that gene may be used as a probe to identify corresponding genes from other species (orthologs) or related genes from the same species. The probes or primers may be used to screen cDNA libraries from various tissue sources believed to express the IL-19 polypeptide. In addition, part or all of a nucleic acid molecule having the sequence as set forth in SEQ ID NO: 5 may be used to screen a genomic library to identify and isolate a gene encoding the amino acid sequence of an IL-19 polypeptide. Typically, conditions of moderate or high stringency will be employed for screening to minimize the number of false positives obtained from the screen.

Nucleic acid molecules encoding the amino acid sequence of IL-19 polypeptides may also be identified by expression cloning which employs the detection of positive clones based upon a property of the expressed protein. Typically, nucleic acid libraries are screened by the binding of an antibody or other binding partner (e.g., receptor or ligand) to cloned proteins which are expressed and displayed on a host cell surface. The antibody or binding partner is modified with a detectable label to identify those cells expressing the desired clone.

Recombinant expression techniques conducted in accordance with the descriptions set forth below may be followed to produce these polynucleotides and to express the encoded polypeptides. For example, by inserting a nucleic acid sequence which encodes the amino acid sequence of an IL-19 polypeptide into an appropriate vector, one skilled in the art can readily produce large quantities of the desired nucleotide sequence. The sequences can then be used to generate detection probes or amplification primers. Alternatively, a polynucleotide encoding the amino acid sequence of an IL-19 polypeptide can be inserted into an expression vector. By introducing the expression vector into an appropriate host, the encoded IL-19 polypeptide may be produced in large amounts.

Another method for obtaining a suitable nucleic acid sequence is the polymerase chain reaction (PCR). In this method, cDNA is prepared from poly(A)+RNA or total RNA using the enzyme reverse transcriptase. Two primers, typically complementary to two separate regions of cDNA (oligonucleotides) encoding the amino acid sequence of an IL-19 polypeptide, are then added to the cDNA along with a polymerase such as Taq polymerase, and the polymerase amplifies the cDNA region between the two primers.

Another means of preparing a nucleic acid molecule encoding the amino acid sequence of an IL-19 polypeptide, including a fragment or variant, is chemical synthesis using methods well known to the skilled artisan such as those described by Engels el al., Angew. Chem. Intl. Ed., 28:716-734 (1989). These methods include, inter alia, the phosphotriester, phosphoramidite, and H-phosphonate methods for nucleic acid synthesis. A preferred method for such chemical synthesis is polymer-supported synthesis using standard phosphoramidite chemistry. Typically, the DNA encoding the amino acid sequence of an IL-19 polypeptide will be several hundred nucleotides in length. Nucleic acids larger than about 100 nucleotides can be synthesized as several fragments using these methods. The fragments can then be ligated together to form the full length nucleotide sequence of an IL-19 polypeptide. Usually, the DNA fragment encoding the amino terminus of the polypeptide will have an ATG, which encodes a methionine residue. This methionine may or may not be present on the mature form of the IL-19 polypeptide, depending on whether the polypeptide produced in the host cell is designed to be secreted from that cell. Other methods known to the skilled artisan may be used as well.

In some cases, it may be desirable to prepare nucleic acid molecules encoding IL-19 polypeptide variants. Nucleic acid molecules encoding variants may be produced using site directed mutagenesis, PCR amplification, or other appropriate methods, where the primer(s) have the desired point mutations (see Sambrook et al., supra, and Ausubel et al., for descriptions of mutagenesis techniques). Chemical synthesis using methods described by Engels et al., may also be used to prepare such variants. Other methods known to the skilled artisan may be used as well.

In certain embodiments, nucleic acid variants contain codons which have been altered for the optimal expression of an IL-19 polypeptide in a given host cell. Particular codon alterations will depend upon the IL-19 polypeptide(s) and host cell(s) selected for expression. Such “codon optimization” can be carried out by a variety of methods, for example, by selecting codons which are preferred for use in highly expressed genes in a given host cell. Computer algorithms which incorporate codon frequency tables such as “Ecohigh.cod” for codon preference of highly expressed bacterial genes may be used and are provided by the University of Wisconsin Package Version 9.0, Genetics Computer Group, Madison, Wis. Other useful codon frequency tables include “Celegans₁₃high.cod”, “Celegans_low.cod”, “Drosophila₁₃high.cod”, “Human₁₃high.cod”, “Maize₁₃high.cod”, and “Yeast₁₃high.cod”.

In other embodiments, nucleic acid molecules encode IL-19 variants with conservative amino acid substitutions as described herein, IL-19 variants comprising an addition and/or a deletion of one or more N-linked or O-linked glycosylation sites, IL-19 variants having deletions and/or substitutions of one or more cysteine residues, or IL-19 polypeptide fragments as described herein. In addition, nucleic acid molecules may encode any combination of IL-19 variants, fragments, and fusion polypeptides described herein.

Variations of Murine IL-19 Polynucleotides and Polypeptides

Purified and isolated polynucleotides (e.g., DNA and RNA transcripts, both sense and anti sense strands) encoding murine IL-19 and variants thereof (i.e., deletion, addition or substitution analogs) are described herein. Preferred DNA molecules include cDNA, genomic DNA and wholly or partially chemically synthesized DNA molecules. A murine IL-19 polynucleotide is the DNA as set forth in SEQ ID NO: 5 encoding the polypeptide of SEQ ID NO: 6. Also contemplated are DNA molecules which hybridize under stringent conditions to the protein coding portion of the DNA of SEQ. ID NO.: 1 and DNA molecules which are at least 85%, at least 90%, at least 95%, at least 96%, at least 97%, at least 98% or at least 99% percent homologous to the polypeptide coding region sequence set out in SEQ. ID NO.: 1. Further contemplated are anti-sense polynucleotides which specifically hybridize to a polynucleotide encoding the amino acid sequence set out in SEQ. ID NO.: 2.

Also provided are recombinant plasmid and viral expression constructs comprising polynucleotides of murine IL-19. Prokaryotic or eukaryotic host cells transformed or transfected with polynucleotides of the invention are contemplated, along with methods for producing an IL-19 polypeptide comprising growing the host cell in a suitable medium under conditions which permit expression of the polypeptide.

Host cells of the invention include any cell type capable of expressing IL-19 and IL-19 binding proteins. In a preferred embodiment, the host cells are of either mammal, insect or yeast origin. In another aspect, the host cell is a yeast cell, selected from various strains, including S. cerevisiae, S.pombe, K.lactis, P.pastoris, S.carlsbergensis and C.albicans. Mammalian host cells of the invention include Chinese hamster ovary (CHO), COS, HeLa, 3T3, CV1, LTK, 293T3, Rat1, PC12 or any other cell line of human or rodent origin routinely used in the art. Insect host cell lines include SF9 cells. Additional plasmids and host cells available for use are described below.

Also provided are purified and isolated murine IL-19 polypeptides, fragments and variants thereof. A preferred IL-19 polypeptide is as set forth in SEQ ID NO: 6. IL-19 products of the invention may be obtained as isolates from natural sources, but, along with IL-19 variant products, are also produced by recombinant procedures using host cells of the invention. Completely glycosylated, partially glycosylated and wholly de-glycosylated forms of the IL-19 polypeptide may be generated by varying the host cell selected for recombinant production and/or post-isolation processing. Variant IL-19 polypeptides of the invention may comprise water soluble and insoluble IL-19 polypeptides and analogs wherein one or more of the amino acids are deleted or replaced: (1) without loss, and preferably with enhancement, of one or more biological activities or immunological characteristics specific for IL-19; or (2) with specific disablement of a particular ligand/receptor binding or signaling function. In one embodiment, the variant or analog IL-19 polypeptides possesses at least 85%, at least 90%, at least 95%, at least 96%, at least 97%, at least 98% or at least 99% percent identity to the amino acid sequence set out in SEQ. ID NO.: 2.

The purified polypeptides can be used in in vitro binding assays which are well known in the art to identify molecules which bind to the polypeptides. These molecules include but are not limited to, for e.g., small molecules, molecules from combinatorial libraries, antibodies or other proteins. The molecules identified in the binding assay are then tested for antagonist or agonist activity in in vivo tissue culture or animal models that are well known in the art. In brief, the molecules are titrated into a plurality of cell cultures or animals and then tested for either cell/animal death or prolonged survival of the animal/cells.

This invention is particularly useful for screening chemical compounds by using the novel polypeptides or binding fragments thereof in any of a variety of drug screening techniques. The polypeptides or fragments employed in such a test may either be free in solution, affixed to a solid support, borne on a cell surface or located intracellularly. One method of drug screening utilizes eukaryotic or prokaryotic host cells which are stably transformed with recombinant nucleic acids expressing the polypeptide or a fragment thereof. Drugs are screened against such transformed cells in competitive binding assays. Such cells, either in viable or fixed form, can be used for standard binding assays. One may measure, for example, the formation of complexes between polypeptides of the invention or fragments and the agent being tested or examine the diminution in complex formation between the novel polypeptides and an appropriate cell line, which are well known in the art.

Sources for test compounds that may be screened for ability to bind to or modulate (i.e., increase or decrease) the activity of polypeptides of the invention include (1) inorganic and organic chemical libraries, (2) natural product libraries, and (3) combinatorial libraries comprised of either random or mimetic peptides, oligonucleotides or organic molecules.

Chemical libraries may be readily synthesized or purchased from a number of commercial sources, and may include structural analogs of known compounds or compounds that are identified as “hits” or “leads” via natural product screening.

The sources of natural product libraries are microorganisms (including bacteria and fungi), animals, plants or other vegetation, or marine organisms, and libraries of mixtures for screening may be created by: (1) fermentation and extraction of broths from soil, plant or marine microorganisms or (2) extraction of the organisms themselves. Natural product libraries include polyketides, non-ribosomal peptides, and (non-naturally occurring) variants thereof. For a review, see Science 282: 63-68 (1998).

Combinatorial libraries are composed of large numbers of peptides, oligonucleotides or organic compounds and can be readily prepared by traditional automated synthesis methods, PCR, cloning or proprietary synthetic methods. Of particular interest are peptide and oligonucleotide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). For reviews and examples of peptidomimetic libraries, see Al-Obeidi et al., Mol. Biotechnol, 9:205-23 (1998); Hruby et al., Curr Opin Chem Biol, 1:114-19 (1997); Domer et al., Bioorg Med Chem, 4:709-15 (1996) (alkylated dipeptides).

Identification of modulators through use of the various libraries described herein permits modification of the candidate “hit” (or “lead”) to optimize the capacity of the “hit” to bind a polypeptide of the invention. The molecules identified in the binding assay are then tested for antagonist or agonist activity in in vivo tissue culture or animal models that are well known in the art. In brief, the molecules are titrated into a plurality of cell cultures or animals and then tested for either cell/animal death or prolonged survival of the animal/cells.

In addition, the peptides of the invention or molecules capable of binding to the peptides may be complexed with toxins, e.g., ricin or cholera, or with other compounds that are toxic to cells. The toxin-binding molecule complex is then targeted to a tumor or other cell by the specificity of the binding molecule for SEQ ID NO.: 6.

Vectors and Host Cells

A nucleic acid molecule encoding the amino acid sequence of an IL-19 polypeptide is inserted into an appropriate expression vector using standard ligation techniques. The vector is typically selected to be functional in the particular host cell employed (i.e., the vector is compatible with the host cell machinery such that amplification of the gene and/or expression of the gene can occur). A nucleic acid molecule encoding the amino acid sequence of an IL-19 polypeptide may be amplified/expressed in prokaryotic, yeast, insect (baculovirus systems), and/or eukaryotic host cells. Selection of the host cell will depend in part on whether an IL-19 polypeptide is to be post-translationally modified (e.g., glycosylated and/or phosphorylated). If so, yeast, insect, or mammalian host cells are preferable. For a review of expression vectors, see Meth. Enz., v. 185, D. V. Goeddel, ed. Academic Press Inc., San Diego, Calif. (1990).

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the IL-19 polypeptide coding sequence; the oligonucleotide sequence encodes polyHis (such as hexaHis), or other “tag” such as FLAG, HA (hemaglutinin Influenza virus) or myc for which commercially available antibodies exist. This tag is typically fused to the polypeptide upon expression of the polypeptide, and can serve as a means for affinity purification of the IL-19 polypeptide from the host cell. Affinity purification can be accomplished, for example, by column chromatography using antibodies against the tag as an affinity matrix. Optionally, the tag can subsequently be removed from the purified IL-19 polypeptide by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source) or synthetic, or the flanking sequences may be native sequences which normally function to regulate IL-19 polypeptide expression. As such, the source of a flanking sequence may be any prokaryotic or eukaryotic organism, any vertebrate or invertebrate organism, or any plant, provided that the flanking sequences are functional in, and can be activated by, the host cell machinery.

The flanking sequences useful in the vectors of this invention may be obtained by any of several methods well known in the art. Typically, flanking sequences useful herein other than the endogenous IL-19 gene flanking sequences will have been previously identified by mapping and/or by restriction endonuclease digestion and can thus be isolated from the proper tissue source using the appropriate restriction endonucleases. In some cases, the full nucleotide sequence of a flanking sequence may be known.

Where all or only a portion of the flanking sequence is known, it may be obtained using PCR and/or by screening a genomic library with suitable oligonucleotide and/or flanking sequence fragments from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. Amplification of the vector to a certain copy number can, in some cases, be important for the optimal expression of an IL-19 polypeptide. If the vector of choice does not contain an origin of replication site, one may be chemically synthesized based on a known sequence, and ligated into the vector. For example, the origin of replication from the plasmid pBR322 (Product No. 303-3s, New England Biolabs, Beverly, Mass.) is suitable for most Gram-negative bacteria and various origins (e.g., SV40, polyoma, adenovirus, vesicular stomatitus virus (VSV) or papillomaviruses such as HPV or BPV) are useful for cloning vectors in mammalian cells. Generally, the origin of replication component is not needed for mammalian expression vectors (for example, the SV40 origin is often used only because it contains the early promoter).

A transcription termination sequence is typically located 3′ of the end of a polypeptide coding region and serves to terminate transcription. Usually, a transcription termination sequence in prokaryotic cells is a G-C rich fragment followed by a poly T sequence. While the sequence is easily cloned from a library or even purchased commercially as part of a vector, it can also be readily synthesized using methods for nucleic acid synthesis such as those described herein.

A selectable marker gene element encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells, (b) complement auxotrophic deficiencies of the cell; or (c) supply critical nutrients not available from complex media. Preferred selectable markers are the kanamycin resistance gene, the ampicillin resistance gene, and the tetracycline resistance gene. A neomycin resistance gene may also be used for selection in prokaryotic and eukaryotic host cells.

Other selection genes may be used to amplify the gene which will be expressed. Amplification is the process wherein genes which are in greater demand for the production of a protein critical for growth are reiterated in tandem within the chromosomes of successive generations of recombinant cells. Examples of suitable selectable markers for mammalian cells include dihydrofolate reductase (DHFR) and thymidine kinase. The mammalian cell transformants are placed under selection pressure which only the transformants are uniquely adapted to survive by virtue of the selection gene present in the vector. Selection pressure is imposed by culturing the transformed cells under conditions in which the concentration of selection agent in the medium is successively changed, thereby leading to the amplification of both the selection gene and the DNA that encodes an IL-19 polypeptide. As a result, increased quantities of IL-19 polypeptide are synthesized from the amplified DNA.

A ribosome binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of an IL-19 polypeptide to be expressed. The Shine-Dalgarno sequence is varied but is typically a polypurine (i.e., having a high A-G content). Many Shine-Dalgarno sequences have been identified, each of which can be readily synthesized using methods set forth herein and used in a prokaryotic vector.

A leader, or signal, sequence may be used to direct an IL-19 polypeptide out of the host cell. Typically, a nucleotide sequence encoding the signal sequence is positioned in the coding region of an IL-19 nucleic acid molecule, or directly at the 5′ end of an IL-19 polypeptide coding region. Many signal sequences have been identified, and any of those that are functional in the selected host cell may be used in conjunction with an IL-19 nucleic acid molecule. Therefore, a signal sequence may be homologous (naturally occurring) or heterologous to an IL-19 gene or cDNA. Additionally, a signal sequence may be chemically synthesized using methods described herein. In most cases, the secretion of an IL-19 polypeptide from the host cell via the presence of a signal peptide will result in the removal of the signal peptide from the secreted IL-19 polypeptide. The signal sequence may be a component of the vector, or it may be a part of an IL-19 nucleic acid molecule that is inserted into the vector.

Included within the scope of this invention is the use of either a nucleotide sequence encoding a native IL-19 polypeptide signal sequence joined to an IL-19 polypeptide coding region or a nucleotide sequence encoding a heterologous signal sequence joined to an IL-19 polypeptide coding region. The heterologous signal sequence selected should be one that is recognized and processed, i.e., cleaved by a signal peptidase, by the host cell. For prokaryotic host cells that do not recognize and process the native IL-19 polypeptide signal sequence, the signal sequence is substituted by a prokaryotic signal sequence selected, for example, from the group of the alkaline phosphatase, penicillinase, or heat-stable enterotoxin II leaders. For yeast secretion, the native IL-19 polypeptide signal sequence may be substituted by the yeast invertase, alpha factor, or acid phosphatase leaders. In mammalian cell expression the native signal sequence is satisfactory, although other mammalian signal sequences may be suitable.

In some cases, such as where glycosylation is desired in a eukaryotic host cell expression system, one may manipulate the various presequences to improve glycosylation or yield. For example, one may alter the peptidase cleavage site of a particular signal peptide, or add presequences, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the N-terminus. Alternatively, use of some enzyme cleavage sites may result in a slightly truncated form of the desired IL-19 polypeptide, if the enzyme cuts at such area within the mature polypeptide.

In many cases, transcription of a nucleic acid molecule is increased by the presence of one or more introns in the vector; this is particularly true where a polypeptide is produced in eukaryotic host cells, especially mammalian host cells. The introns used may be naturally occurring within the IL-19 gene, especially where the gene used is a full length genomic sequence or a fragment thereof. Where the intron is not naturally occurring within the gene (as for most cDNAs), the intron(s) may be obtained from another source. The position of the intron with respect to flanking sequences and the IL-19 gene is generally important, as the intron must be transcribed to be effective. Thus, when an IL-19 cDNA molecule is being transcribed, the preferred position for the intron is 3′ to the transcription start site, and 5′ to the polyA transcription termination sequence. Preferably, the intron or introns will be located on one side or the other (i.e., 5′ or 3′) of the cDNA such that it does not interrupt the coding sequence. Any intron from any source, including any viral, prokaryotic and eukaryotic (plant or animal) organisms, may be used to practice this invention, provided that it is compatible with the host cell(s) into which it is inserted. Also included herein are synthetic introns. Optionally, more than one intron may be used in the vector.

The expression and cloning vectors of the present invention will each typically contain a promoter that is recognized by the host organism and operably linked to the molecule encoding a IL-19 polypeptide. Promoters are untranscribed sequences located upstream (5′) to the start codon of a structural gene (generally within about 100 to 1000 bp) that control the transcription of the structural gene. Promoters are conventionally grouped into one of two classes, inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no control over gene expression. A large number of promoters, recognized by a variety of potential host cells, are well known. A suitable promoter is operably linked to the DNA encoding an IL-19 polypeptide by removing the promoter from the source DNA by restriction enzyme digestion and inserting the desired promoter sequence into the vector. The native IL-19 gene promoter sequence may be used to direct amplification and/or expression of an IL-19 nucleic acid molecule. A heterologous promoter is preferred, however, if it permits greater transcription and higher yields of the expressed protein as compared to the native promoter, and if it is compatible with the host cell system that has been selected for use.

Promoters suitable for use with prokaryotic hosts include the beta-lactamase and lactose promoter systems; alkaline phosphatase, a tryptophan (trp) promoter system; and hybrid promoters such as the tac promoter. Other known bacterial promoters are also suitable. Their sequences have been published, thereby enabling one skilled in the art to ligate them to the desired DNA sequence(s), using linkers or adapters as needed to supply any useful restriction sites.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus (CMV), a retrovirus, hepatitis-B virus and most preferably Simian Virus 40 (SV40). Other suitable mammalian promoters include heterologous mammalian promoters, e.g., heat-shock promoters and the actin promoter.

Additional promoters which may be of interest in controlling IL-19 gene transcription include, but are not limited to: the SV40 early promoter region (Bernoist and Chambon, Nature, 290:304-310, 1981); the CMV promoter; the promoter contained in the 3′ long terminal repeat of Rous sarcoma virus (Yamamoto et al., Cell, 22:787-797, 1980); the herpes thymidine kinase promoter (Wagner et al., Proc. Natl. Acad. Sci. USA, 78:144-1445, 1981); the regulatory sequences of the metallothionine gene (Brinster et al., Nature, 296:39-42, 1982); prokaryotic expression vectors such as the beta-lactamase promoter (Villa-Kamaroff, et al., Proc. Natl. Acad. Sci. USA, 75:3727-3731, 1978); or the tac promoter (DeBoer, et al., Proc. Natl. Acad. Sci. USA, 80:21-25, 1983). Also of interest are the following animal transcriptional control regions, which exhibit tissue specificity and have been utilized in transgenic animals: the elastase I gene control region which is active in pancreatic acinar cells (Swift et al., Cell, 38:639-646, 1984; Omitz et al., Cold Spring Harbor Symp. Quant. Biol., 50:399-409 (1986); MacDonald, Hepatology, 7:425-515, 1987); the insulin gene control region which is active in pancreatic beta cells (Hanahan, Nature, 315:115-122, 1985); the immunoglobulin gene control region which is active in lymphoid cells (Grosschedl et al., Cell, 38:647-658 (1984); Adames et al., Nature, 318:533-538 (1985); Alexander et al., Mol. Cell. Biol., 7:1436-1444, 1987); the mouse mammary tumor virus control region which is active in testicular, breast, lymphoid and mast cells (Leder et al., Cell, 45:485-495, 1986); the albumin gene control region which is active in liver (Pinkert et al., Genes and Devel., 1:268-276, 1987); the alphafetoprotein gene control region which is active in liver (Krumlauf et al., Mol. Cell. Biol., 5:1639-1648, 1985; Hammer et al., Science, 235:53-58, 1987); the alpha 1-antitrypsin gene control region which is active in the liver (Kelsey et al., Genes and Devel., 1:161-171, 1987); the beta-globin gene control region which is active in myeloid cells (Mogram et al., Nature, 315:338-340, 1985; Kollias et al., Cell, 46:89-94, 1986); the myelin basic protein gene control region which is active in oligodendrocyte cells in the brain (Readhead et al., Cell, 48:703-712, 1987); the myosin light chain-2 gene control region which is active in skeletal muscle (Sani, Nature, 314:283-286, 1985); and the gonadotropic releasing hormone gene control region which is active in the hypothalamus (Mason et al., Science, 234:1372-1378, 1986).

Expression vectors of the invention may be constructed from a starting vector such as a commercially available vector. Such vectors may or may not contain all of the desired flanking sequences. Where one or more of the desired flanking sequences are not already present in the vector, they may be individually obtained and ligated into the vector. Methods used for obtaining each of the flanking sequences are well known to one skilled in the art.

Preferred vectors for practicing this invention are those which are compatible with bacterial, insect, and mammalian host cells. Such vectors include, inter alia, pCRII, pCR3, and pcDNA3.1 (Invitrogen Company, Carlsbad, Calif.), pBSII (Stratagene Company, La Jolla, Calif.), pET15(Novagen, Madison, Wis.), pGEX (Pharmacia Biotech, Piscataway, N.J.), pEGFP-N2 (Clontech, Palo Alto, Calif.), pETL (BlueBacII; Invitrogen), pDSR-alpha (PCT Publication No. WO90/14363) and pFastBacDual (Gibco/BRL, Grand Island, N.Y.).

Additional suitable vectors include, but are not limited to, cosmids, plasmids or modified viruses, but it will be appreciated that the vector system must be compatible with the selected host cell. Such vectors include, but are not limited to plasmids such as Bluescript® plasmid derivatives (a high copy number ColE 1-based phagemid, Stratagene Cloning Systems Inc., La Jolla Calif.), PCR cloning plasmids designed for cloning Taq-amplified PCR products (e.g., TOPO™ TA Cloning® Kit, PCR2.1® plasmid derivatives, Invitrogen, Carlsbad, Calif.), and mammalian, yeast, or virus vectors such as a baculovirus expression system (pBacPAK plasmid derivatives, Clontech, Palo Alto, Calif.). The recombinant molecules can be introduced into hose cells via transformation, transfection, infection. Electroporation, or other known techniques.

After the vector has been constructed and a nucleic acid molecule encoding an IL-19 polypeptide has been inserted into the proper site of the vector, the completed vector may be inserted into a suitable host cell for amplification and/or polypeptide expression. The transformation of an expression vector for an IL-19 polypeptide into a selected host cell may be accomplished by well known methods including methods such as transfection, infection, calcium chloride, electroporation, microinjection, lipofection or the DEAE-dextran method or other known techniques. The method selected will in part be a function of the type of host cell to be used. These methods and other suitable methods are well known to the skilled artisan, and are set forth, for example, in Sambrook et al., supra.

Host cells may be prokaryotic host cells (such as E. coli) or eukaryotic host cells (such as a yeast cell, an insect cell or a vertebrate cell). The host cell, when cultured under appropriate conditions, synthesizes an IL-19 polypeptide which can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity, such as glycosylation or phosphorylation, and ease of folding into a biologically active molecule.

A number of suitable host cells are known in the art and many are available from the American Type Culture Collection (ATCC), 10801 University Boulevard, Manassas, Va. 20110-2209. Examples include, but are not limited to, mammalian cells, such as Chinese hamster ovary cells (CHO) (ATCC No. CCL61) CHO DHFR-cells (Urlaub et al., Proc. Natl. Acad. Sci. USA, 97:4216-4220 (1980)), human embryonic kidney (HEK) 293 or 293T cells (ATCC No. CRL1573), or 3T3 cells (ATCC No. CCL92). The selection of suitable mammalian host cells and methods for transformation, culture, amplification, screening and product production and purification are known in the art. Other suitable mammalian cell lines, are the monkey COS-1 (ATCC No. CRL1650) and COS-7 cell lines (ATCC No. CRL1651), and the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Candidate cells may be genotypically deficient in the selection gene, or may contain a dominantly acting selection gene. Other suitable mammalian cell lines include but are not limited to, mouse neuroblastoma N2A cells, HeLa, mouse L-929 cells, 3T3 lines derived from Swiss, Balb-c or NIH mice, BHK or HaK hamster cell lines, which are available from the ATCC. Each of these cell lines is known by and available to those skilled in the art of protein expression.

Use of Nucleic Acids as Probes

Another aspect of the subject invention is to provide for polypeptide-specific nucleic acid hybridization probes capable of hybridizing with naturally occurring nucleotide sequences. The hybridization probes of the subject invention may be derived from any of the nucleotide sequences of SEQ ID NO.: 1. Any suitable hybridization technique can be employed, such as, for example, in situ hybridization. PCR as described in U.S. Pat. Nos. 4,683,195 and 4,965,188 provides additional uses for oligonucleotides based upon the nucleotide sequences. Such probes used in PCR may be of recombinant origin, may be chemically synthesized, or a mixture of both. The probe will comprise a discrete nucleotide sequence for the detection of identical sequences or a degenerate pool of possible sequences for identification of closely related genomic sequences.

Other means for producing specific hybridization probes for nucleic acids include the cloning of nucleic acid sequences into vectors for the production of mRNA probes. Such vectors are known in the art and are commercially available and may be used to synthesize RNA probes in vitro by means of the addition of the appropriate RNA polymerase as T7 or SP6 RNA polymerase and the appropriate radioactively labeled nucleotides. The nucleotide sequences may be used to construct hybridization probes for mapping their respective genomic sequences. The nucleotide sequence provided herein may be mapped to a chromosome or specific regions of a chromosome using well-known genetic and/or chromosomal mapping techniques. These techniques include in situ hybridization, linkage analysis against known chromosomal markers, hybridization screening with libraries or flow-sorted chromosomal preparations specific to known chromosomes, and the like. The technique of fluorescent in situ hybridization of chromosome spreads has been described, among other places, in Verma et al (1988) Human Chromosomes: A Manual of Basic Techniques, Pergamon Press, New York N.Y.

Fluorescent in situ hybridization of chromosomal preparations and other physical chromosome mapping techniques may be correlated with additional genetic map data. Examples of genetic map data can be found in the 1994 Genome Issue of Science (265:1981f). Correlation between the location of a nucleic acid on a physical chromosomal map and a specific disease (or predisposition to a specific disease) may help delimit the region of DNA associated with that genetic disease. The nucleotide sequences of the subject invention may be used to detect differences in gene sequences between normal, carrier or affected individuals.

Conditions for incubating a nucleic acid probe or antibody with a test sample vary. Incubation conditions depend on the format employed in the assay, the detection methods employed, and the type and nature of the nucleic acid probe or antibody used in the assay. One skilled in the art will recognize that any one of the commonly available hybridization, amplification or immunological assay formats can readily be adapted to employ the nucleic acid probes or antibodies of the present invention. Examples of such assays can be found in Chard, T., An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of immunoassays: Laboratory Techniques in Biochemistry and Molecular Biology, Elsevier Science Publishers, Amsterdam, The Netherlands (1985). The test samples of the present invention include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are well known in the art and can be readily be adapted in order to obtain a sample which is compatible with the system utilized.

An on-chip strategy for the preparation of DNA probe for the preparation of DNA probe arrays may be employed. For example, addressable laser-activated photodeprotection may be employed in the chemical synthesis of oligonucleotides directly on a glass surface, as described by Fodor et al. (1991) Science 251: 767-73, incorporated herein by reference. Probes may also be immobilized on nylon supports as described by Van Ness et al. (1991) Nucleic Acids Res. 19: 3345-50; or linked to Teflon using the method of Duncan & Cavalier (1988) Anal Biochem 169: 104-8; all references being specifically incorporated herein.

One particular way to prepare support bound oligonucleotides is to utilize the light-generated synthesis described by Pease et al., (1994) Proc. Natl. Acad. Sci USA 91: 5022-6. These authors used current photolithographic techniques to generate arrays of immobilized oligonucleotide probes (DNA chips). These methods, in which light is used to direct the synthesis of oligonucleotide probes in high-density, miniaturized arrays, utilize photolabile 5′-protected N-acyl-deoxynucleoside phosphoramidites, surface linker chemistry and versatile combinatorial synthesis strategies. A matrix of 256 spatially defined oligonucleotide probes may be generated in this manner.

Identification of Polymorphisms

The demonstration of polymorphisms makes possible the identification of such polymorphisms in human subjects and the pharmacogenetic use of this information for diagnosis and treatment. Such polymorphisms may be associated with, e.g., differential predisposition or susceptibility to various disease states (such as disorders involving inflammation or immune response) or a differential response to drug administration, and this genetic information can be used to tailor preventive or therapeutic treatment appropriately. For example, the existence of a polymorphism associated with a predisposition to inflammation or autoimmune disease makes possible the diagnosis of this condition in humans by identifying the presence of the polymorphism.

Polymorphisms can be identified in a variety of ways known in the art which all generally involve obtaining a sample from a patient, analyzing DNA from the sample, optionally involving isolation or amplification of the DNA, and identifying the presence of the polymorphism in the DNA. For example, PCR may be used to amplify an appropriate fragment of genomic DNA which may then be sequenced. Alternatively, the DNA may be subjected to allele-specific oligonucleotide hybridization (in which appropriate oligonucleotides are hybridized to the DNA under conditions permitting detection of a single base mismatch) or to a single nucleotide extension assay (in which an oligonucleotide that hybridizes immediately adjacent to the position of the polymorphism is extended with one or more labeled nucleotides). In addition, traditional restriction fragment length polymorphism analysis (using restriction enzymes that provide differential digestion of the genomic DNA depending on the presence or absence of the polymorphism) may be performed. Arrays with nucleotide sequences of the present invention can be used to detect polymorphisms. The array can comprise modified nucleotide sequences of the present invention in order to detect the nucleotide sequences of the present invention. In the alternative, any one of the nucleotide sequences of the present invention can be placed on the array to detect changes from those sequences.

Alternatively a polymorphism resulting in a change in the amino acid sequence could also be detected by detecting a corresponding change in amino acid sequence of the protein, e.g., by an antibody specific to the variant sequence.

The following examples are intended to be using procedures such as those described in the following examples, some of which are prophetic. The examples assist in further describing the invention, but are not intended in any way to limit the scope of the invention.

### EXAMPLE 1

### Identification of Human Genomic IL-19

Single nucleotide polymorphisms (SNPs) in the IL-10 promoter region have been implicated as a potential cause for several autoimmune diseases and other conditions involving dysregulation of IL-10 activity and function. Due to the importance of the promoter region in regulating cytokine activity, it was necessary to identify the location of the IL-19 promoter.

A homology screening of the NCBI human high throughput genome database (http://www.ncbi.nlm.nih.gov) using the human IL-19 cDNA sequences as a query was carried out using a basic Blast search. The human genomic clone (clone ID: RP11-237C22) was identified (accession number AF276915) and purchased from Research Genentics Inc. (Huntsville, Ala.). The genomic DNA was isolated from the BAC clone and used in the PCR amplification of the promoter fragments.

Full-length human IL-19 was obtained by repetitive 5′ Rapid Amplification of cDNA End (RACE) from genomic clone (clone ID RP11-237C22) using anchor primers and the gene specific antisense primers:

5′-gatatagctgattaatca-3′(RT primer) (SEQ. ID NO.: 2), 5′-taaactccccatctccatgcaa-3′(1st PCR) (SEQ. ID NO.: 3) 5′-caattctatgtccatgcagaaaaat-3′ (2nd PCR) (SEQ. ID NO.: 4). The 5′-end of untranslated sequences of the human cDNA was obtained by a series of repeated 5′ RACE. After three rounds of 5′ RACE, the 5′ end of exon 1 was determined. After obtaining the full-length cDNA clone, the cDNA sequence was compared with the human genomic sequences to locate the exon/intron boundaries. The locations of the introns in this region are found at nucleotide −690 and nucleotide −3.

Gallagher et al. (supra) initially showed that human IL-19 consists of five exons and four introns, and also identified another longer-form transcript containing an alternative translation start site which is in-frame with the rest of the IL-19 mRNA, and predicted that there is one intron near the initiating Met. In the present study, 5′ RACE results revealed two additional exons and two introns in the 5′ untranslated region. Therefore, human IL-19 gene contains seven exons and six introns. The exon/intron junctions conform to the GT/AT rule. The human IL-19 protein is encoded by exon 3 to exon 7.

During the process of isolating the 5′ untranslated region, we also found another alternatively spliced variant in which the first exon ends at nucleotide −849 and the second exon begins at nucleotide −690. This transcript variant, therefore, has a longer intron, 4752 base pairs (bp) instead of 4593 base pairs.

### EXAMPLE 2

### Promoter Activity of Human IL-19

To characterize the DNA sequences involved in the human IL-19 gene expression, five potential promoter fragments (A, B, C, D, and E) were amplified by PCR using a human genomic clone as a template.

Five different regions upstream of exon 1 of the human IL-19 gene were amplified by PCR from the DNA of the BAC clone RP 11-237C22. Five fragments (pA, pB, pC, pD, pE) containing different lengths of sequences upstream of exon 1 and 246 bp (−693 to −939) of exon 1 were ligated into the vector of the promoterless luciferase gene (pGL3 enhancer). pA contains 2104 bp (from −693 to −2907). pB contains 1364 bp (from −2057 to −693). pC contains 1084 bp (from −1777 to −693). pD contains 712 bp (from −1405 to −693). pE contains 393 bp (from −1086 to −693). The sizes of the PCR fragments ranged from 2.1 kb to 393 bp upstream of exon 1.

Five fusion genes (pA, pB, pC, pD, and pE.)were generated by cloning these fragments into the Sac I-Xho I site of the pGL3 enhancer plasmid vector containing the entire coding sequences of firefly luciferase and SV40 enhancer (Promega Corp., Madison, Wis.).

During isolation of the full-length cDNA clone, partial cDNA sequences from human kidney RNA were isolated. Northern blot analysis of kidney tissue showed expression of IL-19 mRNA, therefore, the canine kidney epithelial-like MDCK cells and human embryonic kidney 293-cells were used for the analysis of promoter activity.

pGL3 enhancer plasmids encoding the fusion genes were transfected into canine kidney epithelial-like MDCK cells and human embryonic kidney 293 cells. Cells at a density of 3×105/well in a 6-well plate were transfected with 1 μg of plasmid DNA from the fusion gene and 0.4 μg of the β-galactosidase gene which was used as an internal transfection efficiency control by using 1 μl of LipofectAMINE 2000 reagent (Invitrogen Corporation: Life Technologies, Inc., Carlsbad, Calif.). Twenty-four hours after transfection, the medium was replaced with fresh medium. Forty-eight hours after transfection, the cells were collected and the luciferase activity was analyzed according to the protocol of the luciferase assay system (Promega). To obtain internal control of β-galactosidase (β-gal) gene transfection, the cell lysate was also used for β-gal activity analysis. The luciferase activity from each promoter-fusion gene was divided by β-gal activity to obtain the true representation of luciferase activity from each promoter-luciferase fusion gene.

All five promoter fragments contained at least one or several TATA boxes. All the fusion genes demonstrated some promoter activity, with the pE fusion gene the highest activity, 7- to 8-fold higher than the negative control of the promoterless pGL3 enhancer vector. This experiment was repeated five times with similar results, The luciferase activity in 293-cells was similar to that of MDCK cells. The promoter region 2.1 kb contained several transcription factor binding sites: several copies of keratinocyte-enhancer, TATA box, NF-κB, AP-1, AP-2, E1A-CS, GATA-1, SP-1, P53, and C/EBP. Previous study has shown that IL-19 is inducible by LPS (Gallagher, et al., supra). LPS was added to the transfectants and it was shown that luciferase activity was not inducible by LPS. This could be due to the constitutive expression of IL-19 in kidney cells.

### EXAMPLE 3

### Identification of Individuals With Polymorphisms in the IL-19 Promoter

The identification of the human IL-19 promoter allows for the screening of individuals to detect polymorphisms in the IL-19 promoter region and possibly identify areas of aberrant regulation of IL-19 cytokine production.

To identify polymorphisms in the IL-19 promoter region of an individual, a DNA sample is taken from the individual from either a tissue sample, such as a biopsy, or from a fluid sample, such as peripheral blood. The DNA is isolated from cells in the tissue or fluid sample of the individual using techniques well-known in the art for DNA isolation, see, e.g. Current Protocols in Molecular Biology (John Wiley and Sons, New York, N.Y. 1992) or Qiagen DNA isolation kits (Qiagen, Calif.).

The sequence of the IL-19 promoter set out in SEQ. ID NO.: 1 is then compared to the DNA sequence of the promoter in the individual. In one method, this is carried out in routine restriction enzyme mapping analysis, wherein each set of DNA to be analyzed, both sample and SEQ. ID NO.: 1, is cut with at least one restriction enzyme and the resulting fragments analyzed by gel electrophoresis, separating the restriction fragments based on size. Techniques for restriction cutting and analysis are well-established in the art, see e.g. Current Protocols in Molecular Biology (supra).

The restriction enzymes cut the DNA at known sites in the human IL-19 promoter of SEQ. ID NO.: 1 and which when electrophoresed exhibit a set pattern of restriction fragments. This known restriction map is compared to the restriction map generated by cutting the DNA sample from the individual and subsequent gel electrophoresis. Differences in these fragment analyses indicate that the IL-19 promoter region of the individual contains at least one nucleotide difference from the IL-19 promoter region in SEQ. ID NO.: 1.

The IL-19 promoter of an individual is also compared to the promoter in SEQ. ID NO.: 1 using PCR amplification analysis. Following DNA isolation as outlined above, nucleotide probes designed to amplify the promoter region of SEQ. ID NO.: 1 are used in PCR reactions to amplify the DNA of SEQ. ID NO.: 1 and DNA corresponding to the IL-19 promoter in an individual. PCR product from the amplification of SEQ. ID NO.: 1 exhibits a known fragment size which is then compared to the fragment size generated by amplification of the DNA sample from the individual. The IL-19 promoter region from an individual which contains at least one nucleotide polymorphism will demonstrate an amplification product shorter or longer than the amplification product of the human IL-19 promoter set out in SEQ. ID NO.: 1 due to an alteration in the nucleotide sequence which does not generate the same PCR product as that of SEQ. ID NO.: 1.

The IL-19 promoter of an individual is also compared to the human IL-19 promoter in SEQ. ID NO.: 1 using DNA hybridization analysis. DNA hybridization is carried out under conditions sufficient for detecting a minimum of one nucleotide difference in hybridizing sequences. Exemplary conditions are either highly stringent or moderately stringent conditions. Stringent conditions can include highly stringent conditions (i.e., hybridization to filter-bound DNA in 0.5 M NaHPO4, 7% sodium dodecyl sulfate (SDS), 1 mM EDTA at 65° C., and washing in 0.1× SSC/0.1% SDS at 68° C.), and moderately stringent conditions (i.e., washing in 0.2× SSC/0.1% SDS at 42° C.).

In instances of hybridization of deoxyoligonucleotides, additional exemplary stringent hybridization conditions include washing in 6×SSC/0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

Simple DNA hybridization experiments utilize large fragments of the IL-19 promoter DNA of SEQ. ID NO.: 1 which are hybridized to the DNA from an individual to assess the sequence similarity between the IL-19 promoter in SEQ. ID NO.: 1 and the IL-19 promoter in the individual. These fragments range in size from 20 nucleotides to over 500 nucleotides. The DNA fragment of SEQ. ID NO. 1 is hybridized with either its complement or with the complement of the IL-19 promoter DNA from the individual. Differences in hybridization in the two fragments indicates the individual possesses at least one nucleotide polymorphism in the IL-19 promoter region. Additional DNA hybridization analysis utilizes a series or set of probes which are fragments comprising the IL-19 promoter region set out in SEQ. ID NO.: 1. These fragments are at least 10 nucleotides in length, at least 15 nucleotides in length or at least 20 nucleotides in length. The set of probes are fragments which comprise consecutive, serial sequences of SEQ. ID NO.: 1 or are sets of probes which comprise overlapping fragments of SEQ. ID NO.: 1, wherein the fragments overlap by at least one nucleotide.

Polymorphism analysis is performed by a series of overlapping sequencing reactions as described in Gibson, et al. (J. Immunol. 166: 3915-22. 2001) or as outlined in U.S. Pat. No. 6,428,964 using “tiling” of serial probes, both incorporated herein by reference.

A “tiled sequence” or “tiling” refers to the contiguous hybridization of probes to a target or sample region, whether separated by single-stranded sequence or not. For analysis of polymorphisms within the IL-19 promoter region, a DNA sample is isolated from an individual as described previously. The sample is prepared for hybridization by techniques well-known in the art and hybridized to the sets of probes outlined above. Likewise, the DNA of SEQ. ID NO.: 1 is hybridized with the sets of probes.

For the “tiling” assay, a series of nucleic acid probes complementary to a contiguous region of the IL-19 promoter DNA are exposed to a sample of DNA from an individual . Probes are designed to hybridize to the sample in a contiguous manner to form a duplex comprising the sample and the contiguous probes “tiled” along the target. If a mutation or other alteration exists in the sample, contiguous tiling will be interrupted, producing regions of single-stranded sample in which no duplex exists. For detecting polymorphisms, an agent that degrades single-stranded nucleic acids, such as the enzyme DNA nuclease S1, is added to the sample resulting in only fragments which contained hybridizable DNA. The degradation products are separated by gel electrophoresis or other methods for separating DNA fragments. Identification of one or more single-stranded regions in the sample is indicative of a mutation or other alteration in the target that prevented probe hybridization in that region.

After detection of a mutation, the region, or genetic locus in the target nucleic acid where the mutation is present may be determined by identification of specific probes that failed to hybridize to the target nucleic acid. For example, the hybridization product is cleaved into two separate double-stranded nucleic acids upon treatment with a degradation agent that preferentially degrades single-stranded nucleic acid. The two nucleic acids are separated and sequenced according to methods known in the art. The relative location and identity of the probes that successfully hybridize to the target nucleic acid can then be determined. Through the process of elimination, the one or more probes that failed to hybridize can be identified, as well as their relative position on the target nucleic acid. The genetic locus having a mutation will have a corresponding wild-type that is complementary to the probe that failed to hybridize.

In one embodiment, at least one of the tiling probes comprises a detectable label. Each probe may comprise a different detectable label, permitting the differential detection of the probes (i.e., for example, the different probes may comprise a nucleotide with a different radioactive isotope, a fluorescent tag, or a molecular weight modifying entity). Differential probe labeling allows the identification of the probe that did not anneal to its target in the case of a mutation.

### EXAMPLE 4

### Isolation and Characterization of Mouse IL-19 cDNA

In order to determine a biological role for the IL-19 cytokine, it was first necessary to generate the polypeptide in a useful form, and one that could be used in vivo in functional studies. To accomplish this, the murine homolog of human IL-19 was isolated and purified.

A partial murine cDNA clone was isolated by PCR amplification from mouse embryo cDNA (Clontech, Palo Alto, Calif.). A pair of primers (sense primer: 5′-agagccatccaagctaaggacacctt-3′ SEQ. ID NO.: 7 and antisense primer: 5′-gcattggtggcttcctgcctgcagt-3′ SEQ. ID NO.: 8) designed from human cDNA sequences were used in PCR amplication.

The full-length mouse cDNA clone (about 1 kb in length) was isolated by 5′ Rapid Amplification of cDNA End (RACE) using anchor primers and the gene specific 5′ and 3′ antisense primers. The 3′ untranslated region contained only one ATTTA mRNA destabilizing segment. Hydropathic analysis predicts a hydrophobic signal peptide of 24 amino acids. Beginning with Leu (residue 25), the mature protein, which contains 152 amino acids, has a predicted molecular mass of 14 kDa. Three potential N-linked glycosylation sites were detected in the amino acid sequences, only two of which, NVT and NAT, are identical to those in human IL-19. The third, NCS, is not present in human IL-19. The mature protein contains six cysteines whose positions are identical to those in human IL-19 (amino acids 28, 75, 76, 120, 126, 128).

The amino acid sequences of mouse IL-19 showed 75% similarity and 71% identity with those in human IL-19, and the genomic structure of mouse IL-19 is similar to that of human IL-19. Locations of exon/intron boundaries in the mouse gene are also indicated in FIG. 1.

Expression and Purification of IL-19 Recombinant Protein

To express the recombinant IL-19 in E. coli, an expression vector was constructed that contained a coding region downstream of the fusion protein sequence (thioredoxin). A cDNA clone coding for the human and mouse IL-19 sequences from Leu to His (amino acid 25 to amino acid 176, from Leu 25 to His 170 for murine) was inserted into pET32 EK/LIC (Novagen, Madison, Wis.). The protein was found mostly in the inclusion bodies and was purified to >95% by a series of affinity chromatography and refolding. Before in vitro use, all preparations of IL-19 recombinant protein were found to contain less than 2 ng/ml LPS endotoxin by the detection methods of Limulus amoebocyte lysate (LAL). The human IL-19 was also expressed in the yeast vector of Pichia pastoris and the protein was purified by a series of affinity chromatography.

The predicted molecular weight of mouse IL-19 containing fusion protein (thioredoxin) is about 35 kDa. Treatment of mouse IL-19 fusion protein with enterokinase to cleave off thioredoxin resulted in the disappearance of the 35 kDa band and the formation of a single 17 kDa band on the SDS-PAGE after protein was purified and reduced with β-ME. Recombinant human IL-19 was similarly expressed and showed the same purification pattern as mouse IL-19. The recombinant protein produced from Pichia pastoris showed three bands on SDS-PAGE after affinity chromatography purification. Amino acid determination of the three bands by mass spectrophotometry showed that all three proteins were human IL-19.

### EXAMPLE 5

### Mouse IL-19 Stimulated Monocytes to Produce IL-6 and TNF-α

To determine the effects of mouse IL-19 on the production of cytokines by monocytes, isolated mouse monocytes were incubated with various concentrations of mouse IL-19.

Mouse monocytes were prepared from the spleen of 8- to 10-week-old male mice. Spleen cells were depleted of erythrocytes. Monocytes were allowed to adhere for 30 min at 37° C., 5% CO2. The nonadherent cells were then removed by three washes with warm medium. The monocytes were >95% pure, as determined by Liu's staining, and contained >98% viable cells.

Isolated monocytes (5×10⁶ cells/ml) were cultured for 8 hrs. in a 6-well plate with increasing concentrations of mouse IL-19, after which the level of cytokine production in the supernatant of monocytes was determined by ELISA using cytokine specific ELISA kits (R&D, Minneapolis, Minn.). Results show that monocytes incubated in PBS alone at 37° C. did not produce IL-6 and TNF-α. However, the amount of IL-6 and TNF-α produced by monocytes increased with the addition of mouse IL-19. The increase of these two cytokines was dosage-dependent on IL-19, with approximately 100 pg/ml IL-6 and 400 pg/ml TNF-α detectable after incubation with 100 ng/ml IL-19. The control sample was incubated with PBS only, and exhibited less than 20 pg/ml IL-6 and 50 pg/ml TNF-α. Endotoxin LPS used as a positive control was added at a concentration of 100 ng/ml.

LPS endotoxin can also induce monocytes to produce IL-6 and TNF-α production. To prove that the production of IL-6 and TNF-α from IL-19 treatment was not due to the contamination of LPS endotoxin in the recombinant protein, the IL-19 protein was heat-denatured at 100° C. for 10 min, a condition under which LPS endotoxin cannot be denatured. The heat-denatured protein was added to monocytes to test its biologic activity. The result showed that the heat-denatured protein had lost its activity. Therefore, the activities observed were not due to contamination of the LPS endotoxin. Mouse IL-10 has been shown to be inactive on human monocytes. In contrast, it was shown that that mouse IL-19 protein is active on human monocytes but that human IL-19 is inactive on mouse monocytes. Results show, however, that human IL-19 had the same activity on human monocytes as mouse IL-19 had on mouse monocytes. Culture of humanmonocytes with increasing concentrations of human IL-19 demonstrated a dose depentdent induction of both TNF-α and IL-6 production, with maximum production of approximately 125 pg/ml IL-6 and 260 pg/ml TNF-α detectable after incubation with 100 ng/ml human IL-19.

Detection of Cytokine Transcripts After IL-19 Stimulation of Monocytes

To investigate whether induction of IL-6 and TNF-α was regulated at the transcriptional level, total RNA was isolated from IL-19 or LPS treated monocytes.

Monocytes were treated with mouse IL-19 (100 ng/ml) or LPS (50 ng/ml) for 4 h and total RNA was isolated from the monocytes. RT-PCR was performed with IL-6-or TNF-α-specific primers using total RNA as templates. Amplified PCR fragments were run on gel electrophoresis. Specific primers for β-actin were also used to amplified a PCR fragment and run on gel as an internal control. IL-6 specific primers used were: 5′-tgt gca atg gca att ctg at -3′(sense) (SEQ. ID NO.: 9) and 5′-gga aat tgg ggt agg aag ga-3′(antisense) (SEQ. ID NO.: 10). TNF-α specific primers used were: 5′-ccc caa agg gat gag aag tt-3′(sense) (SEQ. ID NO.: 11) and 5′-gtg ggt gag gag cac gta gt-3′(antisense) (SEQ. ID NO.: 12). Mouse β-actin specific primers used were: 5′-ggg aat ggg tca gaa gga ct-3′(sense) (SEQ. ID NO.: 9) and 5′-ttt gat gtc acg cac gat tt-3′(antisense) (SEQ. ID NO.: 10).

The levels of IL-6 and TNF-α transcripts analyzed by RT-PCR showed that both IL-6 and TNF-α transcripts were induced in monocytes stimulated with IL-19. Induction of IL-6 and TNF-α transcripts may not require de novo protein synthesis because the addition of cycloheximide (0.3 mM added 1 h after IL-19 addition and incubated with cells for another 7 h) did not inhibit the induction.

Effect of IL-10 on IL-19 Cytokine Stimulation

Previous study has shown that IL-10 inhibited IL-6 and TNF-α production in monocytes.

To determine the effects of IL-10 on IL-19 stimulation of IL-6 and TNF-α, isolated monocytes were pretreated with IL-10 (50 ng/ml) or IL-19 (50 ng/ml) for 2 h, and then the other cytokine, either IL-19 or IL-10, was added to the culture. Six hours after co-incubation with both cytokines, monocyte supernatants were collected together with the controls (PBS or single cytokine treatment) and production of IL-6 and TNF-α were measured using cytokine specific ELISA kits according to manufacturer's instructions (R&D, Minneapolis, Minn.).

Results show that pretreatment of monocytes with IL-10 for 2 h followed by IL-19 abolished both IL-6 and TNF-α production by IL-19. However, treatment of monocytes with IL-19 followed by IL-10 only partially blocked IL-19 mediated IL-6 production, while a majority of IL-19 mediated TNF-α production was inhibited by IL-10. This result demonstrated that the interaction of IL-19 with IL-10 exerted differing effects on the production of IL-6 and TNF-α.

Both LPS and IL-19 induced IL-6 and TNF-α. Therefore, we also added LPS and IL-19 together to the monocytes and analyze if both have any synergistic effect. The result demonstrated there was no synergistic effect.

### EXAMPLE 6

### IL-19-Induced Monocyte Apoptosis

An increase in TNF-α levels in the cellular environment is known to augment programmed cell death in affected cell populations. Because IL-19 stimulates TNF-α production by monocytes, cell viability assays were performed to assess the affects of IL-19 on cell death.

During the incubation of monocytes with IL-19, trypan blue staining showed a decrease in cell viability. Cell apoptosis as a result of IL-19 culture was therefore further analyzed using three different methods. Mouse monocytes were treated with IL-19 for 12 h, and then cell viability was measured by propidium iodide (PI) staining, Annexin-V staining, and DNA fragmentation.

Mouse monocytes were treated with PBS or mouse IL-19 (100 ng/ml) alone or in combination with TNF-α antibody for 12 h. After treatment, cells were stained with 1 ml of a solution containing 100 μg/ml propidium iodide (PI) at room temperature for 15 min and then analyzed by flow cytometry (FACScan; Becton Dickinson, Franklin Lakes, N.J.).

Monocytes treated with 100 ng/ml of IL-19 resulted in 33% cell death, while the control showed only 13-16% cell death. LPS endotoxin produced only 23% cell death and heat denatured IL-19 resulted in 17% monocyte death, indicating that culture with IL-19 induced greater cell death than incubation with LPS.

Apoptosis was assessed using an Annexin-V staining kit containing Annexin V fluoroisothiocyanate (FITC) (Clontech). Early in apoptosis, the phosphatidylserine in the inner membrane translocates to the outer surface of the plasma membrane and has a high affinity for Annexin-V, which makes Annexin-V staining an alternative method to demonstrate cell apoptosis. Cells were treated as above, harvested, and then resuspended in 1× binding buffer at a concentration of 1×10⁶ cells/ml. Five μl of Annexin V-FITC was added to 100 μl of the cell solution. The cells were gently vortexed and incubated in the dark for 15 min at room temperature, then analyzed by flow cytometry (FACScan; Becton Dickinson). Treatment of monocytes with mouse IL-19 increased the population of Annexin-V-stained dead cells. LPS endotoxin also induced cell death. This apoptotic effect of IL-19 may be due to the production of TNF-α, because addition of both IL-19 and TNF-α antibody abolished the apoptotic effect of IL-19.

To further verify the apoptotic effect of IL-19, after IL-19 treatment, DNA fragmentation analysis was performed.

Using the method described by Oren and Prives (Biochim. Biophys. Acta 1288:R13. 1996), mouse lymphocytes (5×10⁶ cells/well) were treated with mouse IL-19 for 12 h. After treatment, the culture medium was removed and the cells were washed twice with PBS and harvested. The cells were fixed with 1 ml 70% ethanol. After storage overnight at 4° C., the ethanol was removed and the cells were resuspended in 1 ml phosphate-citric acid buffer with 0.2 M Na2HPO4 and 0.1 M citric acid (pH 7.8) and maintained in this solution at room temperature for 60 min with occasional shaking. This treatment extracts low molecular weight DNA from apoptotic cells but has no effect on the DNA content of nonapoptotic cells (Darzynkiewicz, et al. Cytometry. 13:795. 1992). The cell suspension was centrifuged at 2000 rpm for 5 min. The supernatant containing low molecular weight DNA was collected for analysis of internucleosomal DNA degradation by agarose gel electrophoresis.

The results showed that mouse IL-19 induced DNA fragmentation of monocytes and that the extent of DNA fragmentation was dosage-dependent.

### EXAMPLE 7

### Mouse IL-19 Induced Monocytes to Produce Reactive Oxygen Species (ROS)

Exposure to certain cytokines induces marked transient increases in the intracellular level of ROS. For example, exposure to TNF-α or IL-1β increases intracellular levels of ROS in NIH3T3 fibroblasts, which suggests that ROS may act as signaling intermediates for TNF-α and IL-1β. These highly reactive ROS molecules regulate many important cellular events in response to TNF-α, including transcription factor activation (NF-κB), cellular proliferation, and apoptosis. Mouse IL-19 induced TNF-α production and resulted in cell apoptosis, which may have been mediated through oxygen radicals.

In order to test if this effect was mediated through the production of ROS, 1×10⁶ mouse monocytes were incubated at 37° C. with different concentrations of mouse IL-19 (from 0 to 50 ng/ml) for various times and the ROS activities were determined at the end of the incubation.

Monocytes were collected and resuspended in 0.2 ml PBS. The chemiluminescence (CL) count was measured in a completely dark chamber of the Chemiluminescence Analyzing System. After a 100-sec. background level determination, 0.5 ml of 25 mM luminol in PBS (pH 7.4) was injected into the sample. The CL was monitored continuously for an additional 600 sec.

Monocytes treated with IL-19 for 6 h showed an increase in ROS formation in a dose-dependent manner. When monocytes were treated with IL-19 at the concentration of 25 ng/ml, ROS production increased with time. However, production of ROS decreased rapidly after 12 h incubation.

To analyze whether production of ROS depends on TNF-α, monocytes were treated with both TNF-α antibody and IL-19, and ROS production was monitored. The TNF-α antibody (final concentration 0.2 μg/ml) was added to monocytes 30 min before or after the addition of IL-19 (final concentration 100 ng/ml), or both reagents were added at the same time. After incubation with both reagents for another 30 min, ROS production from monocytes was measured by CL count. Results demonstrated that in monocytes treated with TNF-α for 30 min followed by IL-19 stimulation for another 30 min, ROS production was partially inhibited. However, if monocytes were treated with IL-19 for 30 min followed by incubation with TNF-a antibody, ROS production was not inhibited. If both IL-19 and TNF-α were added at the same time, the extent of inhibition on ROS production was not as great as when TNF-α antibody was added first. These results indicate that ROS production may not be completely dependent on TNF-α production.

### EXAMPLE 8

### Animal Models for Determining IL-19 Therapeutic Utility

Several studies demonstrate that IL-19 is expressed primarily by activated monocytes/macrophages. In mouse, SK39-positive macrophages have been identified in splenic red pulp where they may participate in the clearance of foreign materials from circulation, and in medulla of lymph nodes [Jutila, et al., J.Leukocyte Biol. 54:30-39 (1993)]. SK39-positive macrophages have also been reported at sites of both acute and chronic inflammation. Furthermore, monocytes recruited to thioglycolate-inflamed peritoneal cavities also express the SK39 antigen. Collectively, these findings suggest that, if SK39+ cells also express IL-19 then these cells participate in inflammation where macrophages play a significant role.

While the function of IL-19 remains unclear, other more well characterized cytokines such as IL-6 and TNF-α have been shown to participate in a wide variety events which lead to upregulation of inflammatory processes. Therefore, it is highly plausible that interfering with the normal IL-19 function may also interfere with inflammation where activated monocytes and macrophages play a significant role. Such an anti-inflammatory effect could result from: i) blocking macrophage recruitment to sites of inflammation, ii) preventing macrophage activation at the site of inflammation or iii) interfering with macrophage effector functions which damage normal host tissue through either specific autoimmune responses or as a result of bystander cell damage.

Disease states in which there is evidence of macrophages playing a significant role in the disease process include multiple sclerosis, arthritis, graft atherosclerosis, some forms of diabetes and inflammatory bowel disease. Animal models, discussed below, have been shown to reproduce many of the aspects of these human disorders. Inhibitors of IL-19 function are tested in these model systems to determine if the potential exists for treating the corresponding human diseases.

Graft Arteriosclerosis

Cardiac transplantation is now the accepted form of therapeutic intervention for some types of end-state heart disease. As the use of cyclosporin A has increased one year survival rates to 80%, the development of progressive graft arteriosclerosis has emerged as the leading cause of death in cardiac transplants surviving beyond the first year. Recent studies have found that the incidence of significant graft arteriosclerosis 3 years following a cardiac transplant is in the range of 36-44% [Adams, et al., Transplantation 53:1115-1119 (1992); Adams, et al., Transplantation 56:794-799 (1993)].

Graft arteriosclerosis typically consists of diffuse, occlusive, intimal lesions which affect the entire coronary vessel wall, and are often accompanied by lipid deposition. While the pathogenesis of graft arteriosclerosis remains unknown, it is presumably linked to histocompatibility differences between donor and recipient, and is immunologic in nature. Histologically, the areas of intimal thickening are composed primarily of macrophages, although T cells are occasionally seen. It is therefore possible that macrophages secreting IL-19 may play a significant role in the induction and/or development of graft arteriosclerosis. In such a case, monoclonal antibodies or small molecule inhibitors (for example, soluble IL-19 receptor polypeptides) of IL-19 function could be given prophylactically to individuals who received heart transplants and are at risk of developing graft arteriosclerosis.

Although atherosclerosis in heart transplants presents the greatest threat to life, graft arteriosclerosis is also seen in other solid organ transplants, including kidneys and livers. Therapeutic use of IL-19 blocking agents could prevent graft arteriosclerosis in other organ transplants and reduce complications resulting from graft failure.

One model for graft arteriosclerosis in the rat involves heterotopic cardiac allografts transplanted across minor histocompatibility barriers. When Lewis cardiac allografts are transplanted into MHC class I and II compatible F-344 recipients, 80% of the allografts survive at least 3 weeks, while 25% of the grafts survive indefinitely. During this low-grade graft rejection, arteriosclerosis lesions form in the donor heart. Arterial lesions in 120 day old allografts typically have diffuse fibrotic intimal thickening indistinguishable in appearance from graft arteriosclerosis lesions found in rejecting human cardiac allografts.

Rats are transplanted with hearts mismatched at minor histocompatibility antigens, for example Lewis into F-344. Monoclonal antibodies specific for rat IL-19 or small molecule inhibitors of IL-19 are given periodically to transplant recipients. Treatment is expected to reduce the incidence of graft arteriosclerosis in non-rejecting donor hearts. Treatment of rats with an inhibitor of IL-19 binding to an IL-19 receptor (e.g. monoclonal antibodies or small molecule inhibitors) may not be limited to prophylactic treatments. Blocking IL-19 function is also be expected to reduce macrophage mediated inflammation and allow reversal of arterial damage in the graft.

Atherosclerosis in Rabbits Fed Cholesterol

Rabbits fed an atherogenic diet containing a cholesterol supplement for approximately 12-16 weeks develop intimal lesions that cover most of the lumenal surface of the ascending aorta [Rosenfeld, et al., Arteriosclerosis 7:9-23 (1987); Rosenfeld, et al., Arteriosclerosis 7:24-34 (1987)]. The atherosclerotic lesions seen in these rabbits are simmer to those in humans. Lesions contain large numbers of T cells, most of which express CD45RO, a marker associated with memory T cells. Approximately half of the infiltrating T cells also express MHC class II antigen and some express the IL-2 receptor suggesting that many of the cells are in an activated state.

One feature of the atherosclerotic lesions found in cholesterol fed rabbits, but apparently absent in rodent models, is the accumulation of foam cell-rich lesions. Foam cell macrophages are believed to result from the uptake of oxidized low-density lipoprotein (LDL) by specific receptors. Oxidized LDL particles have been found to be toxic for some cell types including endothelial cells and smooth muscle cells. The uptake of potentially toxic, oxidized LDL particles by macrophages serves as an irritant and drives macrophage activation, contributing to the inflammation associated with atherosclerotic lesions.

Once monoclonal antibodies have been generated to rabbit IL-19, cholesterol fed rabbits are treated. Treatments include prophylactic administration of IL-19 monoclonal antibodies or small molecule inhibitors, to demonstrate that IL-19 secreting macrophages are involved in the disease process. Additional studies would demonstrate that monoclonal antibodies to IL-19 or small molecule inhibitors are capable of reversing vessel damage detected in rabbits fed an atherogenic diet.

Insulin-dependent Diabetes

BB rats spontaneously develop insulin-dependent diabetes at 70-150 days of age. Using immunohistochemistry, MHC class II+, ED1+ macrophages can be detected infiltrating the islets early in the disease. Many of the macrophages appear to be engaged in phagocytosis of cell debris or normal cells. As the disease progresses, larger numbers of macrophages are found infiltrating the islets, although significant numbers of T cells, and later B cells, also appear to be recruited to the site [Hanenberg, et al., Diabetologia 32:126-134 (1989)].

Development of diabetes in BB rats appears to depend on both early macrophage infiltration and subsequent T cells recruitment. Treatment of BB rats with silica particles, which are toxic to macrophages, has been effective in blocking the early macrophage infiltration of the islets. In the absence of early macrophage infiltration, subsequent tissue damage by an autoaggressive lymphocyte population fails to occur. Administration of monoclonal antibody OX-19 (specific for rat CD5) or monoclonal antibody OX-8 (specific for rat CD8), which block the T cell-associated phase of the disease, is also effective in suppressing the development of diabetes.

The central role of macrophages and inflammatory cytokines such as IFN-γ and TNF-α in the pathology of this model makes it attractive for testing inhibitors of IL-19 function. Rats genetically predisposed to the development of insulin-dependent diabetes are treated with monoclonal antibodies to IL-19 or small molecule inhibitors and evaluated for the development of the disease. Preventing or delaying clinical onset is evidence that IL-19 plays a role in decreasing the amount of inflammatory cytokine present in the environment thereby decreasing the damage to the islet cells.

Inflammatory Bowel Disease (Crohn's Disease, Ulcerative Colitis)

Animal models used in the study of inflammatory bowel disease (IBD) are generally elicited by intrarectal administration of noxious irritants (e.g. acetic acid or trinitrobenzene sulfonic acid/ethanol). Colonic inflammation induced by these agents is the result of chemical or metabolic injury and lacks the chronic and spontaneously relapsing inflammation associated with human IBD. However, a recently described model using subserosal injections of purified peptidoglycan-polysaccharide (PG-PS) polymers from either group A or group D streptococci appears to be a more physiologically relevant model for human IBD [Yamada, et al., Gastroenterology 104:759-771 (1993)].

In this model PG-PS is injected into the subserosal layer of the distal colon. The resulting inflammatory response is biphasic with an initial acute episode three days after injection, which is followed by a spontaneous chronic phase three to four weeks later. The late phase response is granulomatous in nature, and results in colonic thickening, adhesions, colonic nodules and mucosal lesions. In addition to mucosal injury, PG-PS colitis frequently leads to arthritis anemia and granulomatous hepatitis. The extraintestinal manifestations of the disease make the model attractive for studying Crohn's colitis in that a significant number of patients with active Crohn's disease suffer from arthritic joint disease and hepatobillary inflammation.

Granulomatous lesions are the result of chronic inflammation which leads to the recruitment and subsequent activation of cells of the monocyte/macrophage lineage. Presence of granulomatous lesions in Crohn's disease and the above animal model make this an attractive clinical target for IL-19 monoclonal antibodies or other inhibitors of IL-19 function. Inhibitors of IL-19 function are expected to block the formation of lesions associated with IBD or even reverse tissue damage seen in the disease.

Arthritis

Arthritis appears to be a multi-factorial disease process involving a variety of inflammatory cell types including neutrophils, T lymphocytes and phagocytic macrophages. Although a variety of arthritis models exist, preparations of streptococcal cell wall proteoglycan produce a disorder most similar to the human disease.

In rats, streptococcal cell wall induces inflammation of peripheral joints characterized by repeated episodes of disease progression followed by remission and eventually resulting in joint destruction over a period of several months [Cromartie, et al., J.Exp.Med. 146:1585-1602 (1977); Schwab et al., Infection and Immunity 59:4436-4442 (1991)]. During the chronic phase of the disease, mononuclear phagocytes or macrophages are believed to play a major role in destruction of the synovium. Furthermore, agents which suppress the recruitment of macrophages into the synovium effectively reduce the inflammation and pathology characteristic of arthritis.

A central role for the macrophage and inflammatory cytokines in synovium destruction that leads to arthritis predicts that monoclonal antibodies to IL-19 or inhibitors of IL-19 function may have therapeutic potential in the treatment of this disease. As in other models previously described, IL-19 monoclonal antibodies or small molecule inhibitors administered prophylactically are expected to block or moderate joint inflammation and prevent destruction of the synovium. Agents that interfere with IL-19 function may also moderate ongoing inflammation by preventing the recruitment of additional macrophages to the joint or blocking macrophage activation. The net result would be to reverse ongoing destruction of the joint and facilitate tissue repair.

Multiple Sclerosis

Although pathogenesis of multiple sclerosis (MS) remains unclear, it is generally accepted that the disease is mediated by CD4+T cells which recognize autoantigens in the central nervous system and initiate an inflammatory cascade. The resulting immune response results in the recruitment of additional inflammatory cells, including activated macrophages which contribute to the disease. Experimental autoimmune encephalomyelitis (EAE) is an animal model which reproduces some aspects of MS. Therefore monoclonal antibodies or small molecule inhibitors to IL-19 are likely to be effective in blocking the inflammatory response in EAE. Such agents also have important therapeutic applications in the treatment of MS.

Immune Complex Alveolitis

Alveolar macrophages located in the alveolar ducts, airways, connective tissue, and pleural spaces of the lung represent the lung's first line of defense against inhaled environmental agents. In response to stimulation by agents, including bacterial-derived LPS, IFN-γ and immune complexes, alveolar macrophages release a variety of potent inflammatory mediators, including highly reactive oxygen radicals and nitrogen intermediates. While superoxide anions, hydrogen peroxide and nitric oxide (NO*) have important functions in eradicating pathogens and lysing tumor targets, these agents can have injurious effects on normal tissues.

In a rat model of immune complex alveolitis, NO* release from alveolar macrophages has been shown to mediate much of the lung damage [Mulligan, et al., Proc.Natl.Acad.Sci.(USA) 88:6338-6342 (1991)]. NO* has also been implicated as a mediator in other immune complex mediated injuries including dermal vasculitis [Mulligan, et al., supra] and could potentially play a role in diseases such as glomerulonephritis.

NO* mediated tissue damage is not limited to inflammation involving immune complexes. For example, microglial cell stimulated, by agents such as PMA, LPS or IFN-*, produce NO* at levels capable of killing oligodendrocytes [Merrill, et al., Immunol. 151:2132 (1993)]. Pancreatic islet cells have also been found to be sensitive to NO*, and macrophage release of this mediator has been implicated in the tissue damage which leads to diabetes [Kroncke, et al., BBRC 175:752-758 (1991)]. More recently, it was conclusively demonstrated that NO* release plays a role in endotoxic shock [MacMicking, et al., Cell 81:641-650 (1995)]. When administered lipopolysaccharide (LPS), normal wild-type mice experience a severe, progressive decline in arterial pressure resulting in death. Mice deficient in inducible nitric oxide, however, experience a much less severe decline in arterial pressure in response to LPS, and all survive the treatment. Thus, monoclonal antibodies to IL-19 may be potent anti-inflammatory agents with potential uses in MS, diabetes, lung inflammation and endotoxic shock.

Rat IgG immune complex-induced alveolitis is a widely used experimental model important in understanding acute lung injury. The injury is elicited by instilling anti-bovine serum albumin (BSA) antibodies into lungs via tracheal cannulation, followed by an intravenous injection of BSA. The formation of immune complexes in the microvasculature of the lung leads to complement activation and the recruitment of neutrophils into the lung. Presumably, formation of immune complexes in the lung following extravasation of leukocytes from the blood and subsequent leukocyte movement across lung epithelium. The subsequent release of mediators, including radicals, TNF-α and nitric oxide (NO*), from activated endothelial cells, neutrophils and macrophages which participate in progression of the disease. Pathologic features of the disease include increased vascular permeability leading to edema and the presence of large numbers of erythrocytes and PMNs present in the alveolar spaces.

TNF-alpha has long been viewed as an important mediator in acute lung inflammation, and responsible for the recruitment of inflammatory cells into sites of inflammation, cell activation and tissue damage. As additional proof that IL-19 may prove useful in moderating lung injury, TNF-alpha levels in the bronchoalveolar lavage fluid were evaluated. Treatment with an inhibitor of IL-19 binding to an IL-19 receptor will decrease TNF-α levels and presumably block activation of resident alveolar macrophages during the formation of immune complex alveolitis, and thereby moderates the release of TNF-α and NO*, and reduces subsequent tissue damage caused by these agents.

Mouse Models of Alzheimer's Disease

A transgenic mouse model for the induction and assessment for therapies of Alzheimer's Disease is described in U.S. Pat. No. 5,986,054. Using the mice described therein and other known rodent models of AD (Rhodin et al. Ann N Y Acad Sci. 2000. 903:345-52; Sutton et al. J Submicrosc Cytol Pathol. 1999. 31:313-23 Bjugstad et al. Brain Res. 1998. 8;795:349-57), the effects of an inhibitor of IL-19 binding to an IL-19 receptor on the development of AD and on inflammatory cytokines and reactive oxygen species is assessed. Inhibitors of IL-19 binding to an IL-19 receptor are useful for downregulating the production of damaging TNF-α and oxygen free radicals involved in the progression of Alzheimer's disease.

Alzheimer's Disease (AD) and ROS

The AD brain exhibits evidence for oxygen radical-mediated damage, a situation commonly known as oxidative stress. Much accumulated information indicates that there is an earlier involvement than previously thought for oxidative stress in the pathogenesis of the disease, making this a potential target for therapeutic intervention, especially in subjects at high risk for developing AD (Pratico D. Biochem Pharmacol. 63:563-7. 2002). It has been shown that administration of an anti-oxidant in rat induced AD reduces free radicals neuronal apoptosis and improves memory of subjects animals (Hashimoto et al. J Neurochem. 81:1084-91. 2002). Because experimental results demonstrated that administration of IL-19 increases the production of reactive oxygen species and increases apoptotic cell death, it follows that administration of inhibitors of IL-19 binding to an IL-19 receptor act as useful anti-oxidant compositions to decrease the production of oxygen radicals and cell death, both of which are intimately involved with progression of Alzheimer's disease. Thus, administration of inhibitors of IL-19 binding to an IL-19 receptor are potentially effective therapeutic compounds in the treatment and amelioration of symptoms of AD.

What is claimed is:

1. A method for increasing production of IL-6 comprising the step of administering to an individual in need thereof of amount of IL-19 polypeptide effective to increase production of IL-6.

2. A method for increasing production of TNF-α comprising the step of administering to an individual in need thereof of amount of IL-19 polypeptide effective to increase production of TNF-α.

3. A method for increasing production of reactive oxygen species (ROS) comprising the step of administration to an individual in need thereof an amount of IL-19 polypeptide effective to increase ROS.

4. A method for increasing apoptosis comprising the step of administration to an individual in need thereof an amount of IL-19 polypeptide effective to increase apoptosis.

5. A method for ameliorating a condition associated with decreased levels of IL-6, TNF-α, reactive oxygen species, or apoptosis comprising the step of administering to an individual an amount of IL-19 effective to increase levels of IL-6, TNF-α, reactive oxygen species, or apoptosis.

6. The method of any one of claims 1 through 5 further comprising administering other therapeutic compounds.

7. A method of transmembrane signaling comprising the step of stimulating the IL-20α/β receptor.

8. A method for increasing production of IL-6 in an individual in need thereof comprising the step of stimulating the IL-20α/β receptor effective to increase production of IL-6.

9. A method for increasing production of TNF-α in an individual in need thereof comprising the step of stimulating the IL-20α/β receptor effective to increase production of TNF-α.

10. A method for increasing production of reactive oxygen species in an individual in need thereof comprising the step of stimulating the IL-20α/β receptor effective to increase production of reactive oxygen species.

11. A method for increasing apoptosis in an individual in need thereof comprising the step of stimulating the IL-20α/β receptor effective to increase apoptosis.

12. The method of any one of claims 7 through 11 wherein stimulating is by contact with an IL-19 polypeptide.

13. A method for modulating inflammation comprising the step of administering to an individual in need thereof of amount of an inhibitor of IL-19 binding to an IL-19 receptor effective to modulate inflammation.

14. The method according to claim 13 wherein production of IL-6 is decreased by administering the inhibitor.

15. The method according to claim 13 wherein production of TNF-α is decreased by administering the inhibitor.

16. A method for decreasing production of reactive oxygen species (ROS) comprising the step of administration to an individual in need thereof an amount of an inhibitor of IL-19 binding to an IL-19 receptor effective to decrease ROS.

17. A method for decreasing apoptosis comprising the step of administration to an individual in need thereof an amount of an inhibitor of IL-19 binding to an IL-19 receptor effective to decrease apoptosis.

18. A method of ameliorating a condition associated with increased levels of IL-6, TNF-α, reactive oxygen species, or apoptosis comprising the step of administering to a individual an amount of an inhibitor of IL-19 binding to an IL-19 receptor effective to decrease levels of IL-6, TNF-α, reactive oxygen species, or apoptosis.

19. The method according to any one of claims claim 14 through 18 wherein the inhibitor of IL-19 binding to an IL-19 receptor is selected from the group consisting of an IL-19 blocking antibody, or an antigen binding fragments of an IL-19 blocking antibody, a soluble form of an IL-19 receptor, and an IL-19 receptor antagonist.

20. The method of any one of claims 13 through 18 further comprising administering other therapeutic compounds.

21. A purified and isolated polynucleotide comprising a promoter for a human IL-19 as set out in SEQ. ID NO.: 1.

22. A method of identifying polymorphisms in an IL-19 promoter region of an individual comprising comparing the IL-19 promoter region in the individual to the IL-19 promoter of SEQ. ID NO.: 1, wherein a difference in the nucleotide sequence of the IL-19 promoter is indicative of a polymorphism in the IL-19 promoter region of the individual.

23. A method of claim 22 wherein the comparison is carried out by restriction enzyme analysis, PCR analysis, DNA hybridization analysis.

24. The method of claim 23 wherein the comparison is carried out using DNA hybridization.

25. The method of claim 24 wherein individual IL-19 promoter is hybridized to a set of fragments of SEQ. ID NO.: 1, said fragments consisting of at least 10 nucleotides, at least 15 nucleotides or at least 20 nucleotides.

26. The method of claim 25 wherein the set of fragments overlap by at least one nucleotide.

27. A purified and isolated murine IL-19 polypeptide having the sequence set out in SEQ. ID NO.: 6.

28. A polynucleotide encoding the polypeptide of claim 27.

29. The polynucleotide of claim 28 having an IL-19 protein coding sequence set out in SEQ. ID NO.: 5.

30. A polypeptide encoded by the polynucleotide of claim 29.

31. A purified and isolated murine polynucleotide encoding a murine IL-19 amino acid sequence selected from the group consisting of:
a) a polynucleotide encoding a purified and isolated murine IL-19 polypeptide having the sequence set out in SEQ. ID NO.: 6 wherein the polynucleotide has an IL-19 protein coding sequence set out in SEQ. ID NO.: 5.
b) a polynucleotide which hybridizes under stringent conditions to the protein coding portion of the polynucleotide of a); and
c) a polynucleotide which is at least 85%, 90%, 95%, 96%, 97%, 98% or 99% percent homologous to the polypeptide coding region sequence set out in SEQ. ID NO.: 5.

32. A polypeptide encoded by the polynucleotide of claims 31.

33. A DNA expression construct comprising a polynucleotide according to claim 31.

34. A host cell transformed with a polynucleotide according to claim 31.

35. A method for producing an IL-19 polypeptide comprising growing a host cell according to claim 34 under conditions that permit expression of the IL-19 polypeptide.

36. An antibody specifically immunoreactive with the IL-19 polypeptide of claim 32.

37. An antibody of claim 36 which is a monoclonal antibody.

38. A method for detecting a polypeptide of claim 32 in a sample, comprising:
a) contacting the sample with a compound that binds to and forms a complex with the polypeptide for a period sufficient to form the complex; and
b) detecting the complex, so that if a complex is detected, the polypeptide of claim 27 is detected.

39. A method for identifying a compound that binds to a polypeptide of claim 32, comprising:
a) contacting a compound with the polypeptide of claim 32 under conditions sufficient to form a polypeptide/compound complex; and
b) identifying the compound in the complex.


SEQUENCE LISTING (14 sequences)

SEQ ID NO: 1 - DNA, 2106 bp, Homo sapiens
gaagtgccgttgtgatacgcttatgttggtgggatgggggaaagaaataacaggtttgtatggagtgttatgaaagaattaaatcttaaccttccattggggtaagctgatggaaacaaccatagcaaaggacagactgaatatttttttattctctttatagaaaataacagtaaaaaaaagtattgacataggagaaggaaacaaaaagttgtcaggagttaatgaatagaaatattatttttttctggattttatggtgtttgtgttatttgttagcttttatgaatttgtaatttgttgtaatttctttctaaataagtatttacttttgtctctaattttgtccatctatttttgaattcaattttcaaattcaaaacgcttctctcggccgggcacggtggctcacgcctgtaatcccagcactttgggaggtcgaggagggcggatcacgaggtcaggagatcgagaccatcctggctaacacagtgaaaccccgtctctactaaaaatacaaaaaattagccaggcgaggtggtgggcgcctgtagtcccagctactcgggaggctgaggcaggagaatggcatgaaccccggggggcggagcctgcagtgagccgagatcgtgccactgcactccagcctgggtgacagcgagactccgtctcaaaaaacaaaacaaaacaaaacaaaaaaaacaaaaagaacaaaatgcttctctctgggcctcagctgttccctcatctgtaaatgagaagggtgggccagatgctctttcatgtctggtgtaatgaagccctggagtgggctgcctatgactgcacgcagctgtaccacaccccacctgggtgtctttgggtgaagtacttggtagctccaagtctcatcttccttatccaaaatgatggacacaaaaatagtattgacctcatggaataggtgtgaagatgaaaacagacaatgcatatggatgcttcacacagaccctggggtggcaaatgtgctcagtacttgttagttattagtgtgagtctactcttttagtctatgaattttgttagaggaactcctgcttaccaggcctctggtttaataaaacatgactggagtgacacatttctaagctcaccaccacttataattacagaagattgatggctatataggacatctcccaccaagcctgcagaatgtccagatgtcccaagtacagcccactttactcagagataacgtcaatgagcagactcaagttgaaggattaatggtcactagagcaccaacagcccctacctttagtgagcacatctgcacattccaagtttaatcatagctccttatagtttcttataagcagagatgttcctaaaggacaggggttcctcctcctgctttctgggcatgcctactctctaatggagtagtttccaataaatttgcttctttgtctgtgctccaattctttcctgtgtgagatctaagaacccactcttggggtctagattgggatcctcttttctggcaacatcttgagtatgtgaccatgagaaatgttagaaattggagtgaaaggtacgtaaacatttgaacccaataccattctctggttctcccagaggcacagtaaaaaaaagtattgacatagaagaaggaaacaaaaagttgtcaggagttaaagaataaagatttttttttctggattttgtggtgtttgtggtatttgttagcttttatgatttgtaatttgttgtaatttctctttctaaataaacgtttacttttgtctctaattttgtatttctatttttgaattcaatttattttcccgcagacagggtctcactctgttgcccaggctggagtgcaatggtgaaattatagcagactgcagtcttcaactcctgacctcaagcaattgtcctgcctcctcaacttcctgactacaggtgtgcatgaggactacaggcaggcatgtgccaacacatgcagctttttttttttttttttttcagagatgtggtctcgctttgttgcctacactggtctcaaactcttggcctcaagggatcctcccacctcggcttcccaaagtgcagagatta

SEQ ID NO: 2 - DNA, 18 bp, Artificial sequence
  Synthetic Primer
gatatagctgattaatca

SEQ ID NO: 3 - DNA, 22 bp, Artificial sequence
  Synthetic Primer
taaactccccatctccatgcaa

SEQ ID NO: 4 - DNA, 25 bp, Artificial sequence
  Synthetic Primer
caattctatgtccatgcagaaaaat

SEQ ID NO: 5 - DNA, 1066 bp, Mus musculus
  CDS (1)..(528)
atgaagacacagtgcgcgtctacctggctcctgggcatgacgttgattctctgctcagttcatatctacagtcttaggagatgtctgatttctgtggacatgcgcctcatagaaaagagtttccacgagatcaagagagccatgcaaactaaggacacctttaaaaatgtcaccatcctgtccctggagaacctcaggagcattaagcctggagatgtgtgctgcatgaccaacaacctgctgacattctacagagacagggtgttccaggaccatcaggagagaagccttgaggtcttaaggagaatcagcagcattgccaactctttcctctgcgtgcagaaatctctggagcgatgtcaggtgcacagacaatgtaactgcagtcaggaagccaccaatgcaactaggatcatccatgacaactacaatcagctggaggtctcatctgctgcccttaagtctctaggagaactgaacatacttttagcctggattgacaggaatcatctggaaactcctgcagcctgacacgaaacgcctcgtctgattatctaaataacggactgtgagggttttttttttaattgttgctgttttattttgtttttgtttgtttgttttgtggtcaacagctacttctgaagaagagagagacatggaaagttcacataattatctagaatgagggtcttttctggtaacttgctgatgtggaaggaatcctctcttctgggcttggagcctttcagaaaaacaattgggatttgaatggatctcagctcctggctgaggtctctgctctctgatcccacctgcaatagctaagttggctgctgtggtatttatagcaatatgtggctgttgagagatctcagatatcataggagcaatagacagcccctctaacttattgtaaagcggcttcttccaggcctatgctgtgcaccttgtggtgtgctgtagtgcatattgtactgactgacgtttacgaataaactgtggtttacctatgaggatgaaaaaaaaaaaaaaaaaaaaaaaaaaaaaaaaaaaaaaaaaaaaa

SEQ ID NO: 6 - protein, 176 aa, Mus musculus
MKTQCASTWLLGMTLILCSVHIYSLRRCLISVDMRLIEKSFHEIKRAMQTKDTFKNVTILSLENLRSIKPGDVCCMTNNLLTFYRDRVFQDHQERSLEVLRRISSIANSFLCVQKSLERCQVHRQCNCSQEATNATRIIHDNYNQLEVSSAALKSLGELNILLAWIDRNHLETPAA

SEQ ID NO: 7 - DNA, 26 bp, Artificial sequence
  Synthetic Primer
agagccatccaagctaaggacacctt

SEQ ID NO: 8 - DNA, 25 bp, Artificial sequence
  Synthetic Primer
gcattggtggcttcctgcctgcagt

SEQ ID NO: 9 - DNA, 20 bp, Artificial sequence
  Synthetic Primer
tgtgcaatggcaattctgat

SEQ ID NO: 10 - DNA, 20 bp, Artificial sequence
  Synthetic Primer
ggaaattggggtaggaagga

SEQ ID NO: 11 - DNA, 20 bp, Artificial sequence
  Synthetic Primer
ccccaaagggatgagaagtt

SEQ ID NO: 12 - DNA, 20 bp, Artificial sequence
  Synthetic Primer
gtgggtgaggagcacgtagt

SEQ ID NO: 13 - DNA, 20 bp, Artificial sequence
  Synthetic Primer
gggaatgggtcagaaggact

SEQ ID NO: 14 - DNA, 20 bp, Artificial sequence
  Synthetic Primer
tttgatgtcacgcacgattt